SEQUENCE LISTING (3 sequences)

SEQ ID NO: 1 - DNA, 2987 bp, Homo sapiens
  CDS (20)..(2089)
accactgcggccaccgccaatgaaacgcctcccgctcctagtggttttttccactttgttgaattgttcctatactcaaaattgcaccaagacaccttgtctcccaaatgcaaaatgtgaaatacgcaatggaattgaagcctgctattgcaacatgggattttcaggaaatggtgtcacaatttgtgaagatgataatgaatgtggaaatttaactcagtcctgtggcgaaaatgctaattgcactaacacagaaggaagttattattgtatgtgtgtacctggcttcagatccagcagtaaccaagacaggtttatcactaatgatggaaccgtctgtatagaaaatgtgaatgcaaactgccatttagataatgtctgtatagctgcaaatattaataaaactttaacaaaaatcagatccataaaagaacctgtggctttgctacaagaagtctatagaaattctgtgacagatctttcaccaacagatataattacatatatagaaatattagctgaatcatcttcattactaggttacaagaacaacactatctcagccaaggacaccctttctaactcaactcttactgaatttgtaaaaaccgtgaataattttgttcaaagggatacatttgtagtttgggacaagttatctgtgaatcataggagaacacatcttacaaaactcatgcacactgttgaacaagctactttaaggatatcccagagcttccaaaagaccacagagtttgatacaaattcaacggatatagctctcaaagttttcttttttgattcatataacatgaaacatattcatcctcatatgaatatggatggagactacataaatatatttccaaagagaaaagctgcatatgattcaaatggcaatgttgcagttgcatttttatattataagagtattggtcctttgctttcatcatctgacaacttcttattgaaacctcaaaattatgataattctgaagaggaggaaagagtcatatcttcagtaatttcagtctcaatgagctcaaacccacccacattatatgaacttgaaaaaataacatttacattaagtcatcgaaaggtcacagataggtataggagtctatgtgcattttggaattactcacctgataccatgaatggcagctggtcttcagagggctgtgagctgacatactcaaatgagacccacacctcatgccgctgtaatcacctgacacattttgcaattttgatgtcctctggtccttccattggtattaaagattataatattcttacaaggatcactcaactaggaataattatttcactgatttgtcttgccatatgcatttttaccttctggttcttcagtgaaattcaaagcaccaggacaacaattcacaaaaatctttgctgtagcctatttcttgctgaacttgtttttcttgttgggatcaatacaaatactaataagctcttctgttcaatcattgccggactgctacactacttctttttagctgcttttgcatggatgtgcattgaaggcatacatctctatctcattgttgtgggtgtcatctacaacaagggatttttgcacaagaatttttatatctttggctatctaagcccagccgtggtagttggattttcggcagcactaggatacagatattatggcacaaccaaagtatgttggcttagcaccgaaaacaactttatttggagttttataggaccagcatgcctaatcattcttggtaatctcttggcttttggagtcatcatatacaaagtttttcgtcacactgcagggttgaaaccagaagttagttgctttgagaacataaggtcttgtgcaagaggagccctcgctcttctggtccttctcggcaccacctggatctttgggggtctccatgttgtgcacgcatcagtggttacagcttacctcttcacagtcagcaatgctttccaggggatgttcatttttttattcctgtgtgttttatctagaaagattcaagaagaatattacagattgttcaaaaatgtcccctgttgttttggatgtttaaggtaaacatagagaatggtggataattacaactgcacaaaaataaaaattccaagctgtggatgaccaatgtataaaaatgactcatcaaattatccaattattaactactagacaaaaagtattttaaatcagtttttctgtttatgctataggaactgtagataataaggtaaaattatgtatcatatagatatactatgtttttctatgtgaaatagttctgtcaaaaatagtattgcagatatttggaaagtaattggtttctcaggagtgatatcactgcacccaaggaaagattttctttctaacacgagaagtatatgaatgtcctgaaggaaaccactggcttgatatttctgtgactcgtgttgcctttgaaactagtcccctaccacctcggtaatgagctccattacagaaagtggaacataagagaatgaaggggcagaatatcaaacagtgaaaagggaatgataagatgtattttgaatgaactgttttttctgtagactagctgagaaattgttgacataaaataaagaattgaagaaacacattttaccattttgtgaattgttctgaacttaaatgtccactaaaacaacttagacttctgtttgctaaatctgtttctttttctaatattctaaaaaaaacaaaaaggtttacctccacaaattgaaaaaaaaaaagtgaaaaaaatctgtttctaaggttagactgagatatatactatttccttacttatttcacagattgtgactttggatagttaatcagtaaaatataaatgtgtcaagatataatattgtttatacctatcaatgtaaaaacagtgtaataaagctgaagtattctattaaaaaaaaaaaaaaaaaaaaaaaaaaagggcggccgc

SEQ ID NO: 2 - protein, 690 aa, Homo sapiens
MKRLPLLVVFSTLLNCSYTQNCTKTPCLPNAKCEIRNGIEACYCNMGFSGNGVTICEDDNECGNLTQSCGENANCTNTEGSYYCMCVPGFRSSSNQDRFITNDGTVCIENVNANCHLDNVCIAANINKTLTKIRSIKEPVALLQEVYRNSVTDLSPTDIITYIEILAESSSLLGYKNNTISAKDTLSNSTLTEFVKTVNNFVQRDTFVVWDKLSVNHRRTHLTKLMHTVEQATLRISQSFQKTTEFDTNSTDIALKVFFFDSYNMKHIHPHMNMDGDYINIFPKRKAAYDSNGNVAVAFLYYKSIGPLLSSSDNFLLKPQNYDNSEEEERVISSVISVSMSSNPPTLYELEKITFTLSHRKVTDRYRSLCAFWNYSPDTMNGSWSSEGCELTYSNETHTSCRCNHLTHFAILMSSGPSIGIKDYNILTRITQLGIIISLICLAICIFTFWFFSEIQSTRTTIHKNLCCSLFLAELVFLVGINTNTNKLFCSIIAGLLHYFFLAAFAWMCIEGIHLYLIVVGVIYNKGFLHKNFYIFGYLSPAVVVGFSAALGYRYYGTTKVCWLSTENNFIWSFIGPACLIILGNLLAFGVIIYKVFRHTAGLKPEVSCFENIRSCARGALALLVLLGTTWIFGGLHVVHASVVTAYLFTVSNAFQGMFIFLFLCVLSRKIQEEYYRLFKNVPCCFGCLR

SEQ ID NO: 3 - DNA, 2073 bp, Homo sapiens
atgaaacgcctcccgctcctagtggttttttccactttgttgaattgttcctatactcaaaattgcaccaagacaccttgtctcccaaatgcaaaatgtgaaatacgcaatggaattgaagcctgctattgcaacatgggattttcaggaaatggtgtcacaatttgtgaagatgataatgaatgtggaaatttaactcagtcctgtggcgaaaatgctaattgcactaacacagaaggaagttattattgtatgtgtgtacctggcttcagatccagcagtaaccaagacaggtttatcactaatgatggaaccgtctgtatagaaaatgtgaatgcaaactgccatttagataatgtctgtatagctgcaaatattaataaaactttaacaaaaatcagatccataaaagaacctgtggctttgctacaagaagtctatagaaattctgtgacagatctttcaccaacagatataattacatatatagaaatattagctgaatcatcttcattactaggttacaagaacaacactatctcagccaaggacaccctttctaactcaactcttactgaatttgtaaaaaccgtgaataattttgttcaaagggatacatttgtagtttgggacaagttatctgtgaatcataggagaacacatcttacaaaactcatgcacactgttgaacaagctactttaaggatatcccagagcttccaaaagaccacagagtttgatacaaattcaacggatatagctctcaaagttttcttttttgattcatataacatgaaacatattcatcctcatatgaatatggatggagactacataaatatatttccaaagagaaaagctgcatatgattcaaatggcaatgttgcagttgcatttttatattataagagtattggtcctttgctttcatcatctgacaacttcttattgaaacctcaaaattatgataattctgaagaggaggaaagagtcatatcttcagtaatttcagtctcaatgagctcaaacccacccacattatatgaacttgaaaaaataacatttacattaagtcatcgaaaggtcacagataggtataggagtctatgtgcattttggaattactcacctgataccatgaatggcagctggtcttcagagggctgtgagctgacatactcaaatgagacccacacctcatgccgctgtaatcacctgacacattttgcaattttgatgtcctctggtccttccattggtattaaagattataatattcttacaaggatcactcaactaggaataattatttcactgatttgtcttgccatatgcatttttaccttctggttcttcagtgaaattcaaagcaccaggacaacaattcacaaaaatctttgctgtagcctatttcttgctgaacttgtttttcttgttgggatcaatacaaatactaataagctcttctgttcaatcattgccggactgctacactacttctttttagctgcttttgcatggatgtgcattgaaggcatacatctctatctcattgttgtgggtgtcatctacaacaagggatttttgcacaagaatttttatatctttggctatctaagcccagccgtggtagttggattttcggcagcactaggatacagatattatggcacaaccaaagtatgttggcttagcaccgaaaacaactttatttggagttttataggaccagcatgcctaatcattcttggtaatctcttggcttttggagtcatcatatacaaagtttttcgtcacactgcagggttgaaaccagaagttagttgctttgagaacataaggtcttgtgcaagaggagccctcgctcttctggtccttctcggcaccacctggatctttgggggtctccatgttgtgcacgcatcagtggttacagcttacctcttcacagtcagcaatgctttccaggggatgttcatttttttattcctgtgtgttttatctagaaagattcaagaagaatattacagattgttcaaaaatgtcccctgttgttttggatgtttaaggtaa

(19) United States
(12) Patent Application Publication
(10) Pub. No.: US 2003/0087343 A1
(43) Pub. Date: May 8, 2003

(54) Novel SLGP nucleic acid molecules and uses therefor

(75) Inventor: Fong-Ying Tsai, Newton, MA (US)

Correspondence Address: LAHIVE & COCKFIELD, 28 STATE STREET, BOSTON, MA, 02109, US

(73) Assignee: Millennium Pharmaceuticals, Inc.

(21) Appl. No.: 10/099,052

(22) Filed: Mar. 15, 2002

Related U.S. Application Data
(63) Continuation of application No. 09/163,821, filed on Sep. 30, 1998, now abandoned.

(51) Int. Cl. C07K 14/705; C07H 21/04; C12P 21/02; C12N 5/06

(52) U.S. Cl. 435/69.1; 435/320.1; 435/325; 530/350; 536/23.5

(57) ABSTRACT

The invention provides isolated nucleic acids molecules, designated SLGP nucleic acid molecules, which encode novel GPCR family members. The invention also provides antisense nucleic acid molecules, recombinant expression vectors containing SLGP nucleic acid molecules, host cells into which the expression vectors have been introduced, and nonhuman transgenic animals in which an SLGPgene has been introduced or disrupted. The invention still further provides isolated SLGP proteins, fusion proteins, antigenic peptides and anti-SLGP antibodies. Diagnostic methods utilizing compositions of the invention are also provided.

### BACKGROUND OF THE INVENTION

G-protein coupled receptors (GPCRs) are one of the major classes of proteins that are responsible for transducing a signal within a cell. GPCRs are proteins that have seven transmembrane domains. Upon binding of a ligand to an extracellular portion of a GPCR, a signal is transduced within the cell that results in a change in a biological or physiological property of the cell.

G protein-coupled receptors (GPCRs), along with G-proteins and effectors (intracellular enzymes and channels which are modulated by G-proteins), are the components of a modular signaling system that connects the state of intracellular second messengers to extracellular inputs. These genes and gene-products are potential causative agents of disease (Spiegel et al, J. Clin. Invest. (1993) 92:1119-1125); McKusick and Amberger, (1993) J. Med. Genet. 30:1-26). Specific defects in the rodopsin gene and the V2 vasopressin receptor gene have been shown to cause various forms of autosomal dominant and autosomal recessive retinitis pigmentosa (see Nathans et al., (1992) Annual Rev. Genet. 26:403-424), and nephrogenic diabetes insipidus (Holtzman et al. (1993) Hum. Mol. Genet. 2:1201-1204). These receptors are of critical importance to both the central nervous system and peripheral physiological processes. Evolutionary analyses suggest that the ancestor of these proteins originally developed in concert with complex body plans and nervous systems.

The GPCR protein superfamily now contains over 250 types of paralogues, receptors that represent variants generated by gene duplications or other processes (as opposed to orthologues, the same receptor from different species and homologues, different forms of a receptor isolated from a single organism). The superfamily can be broken down into five families: Family I, receptors typified by rhodopsin and the beta2-adrenergic receptor and currently represented by over 200 unique members (reviewed by Dohlman et al., (1991) Annu. Rev. Biochem. 60:653-688); Family II, the recently characterized parathyroid hormone/calcitonin/secretin receptor family (Juppner et al. (1991) Science 254:1024-1026; Lin et al. (1991) Science 254:1022-1024); Family III, the metabotropic glutamate receptor family in mammals, including GABA receptors (Nakanishi et al. (1992) Science 258: 597-603); Family IV, the cAMP receptor family, important in the chemotaxis and development of D. discoideum (Klein et al.(1988) Science 241:1467-1472); and Family V, the fungal mating pheromone receptors such as STE2 (reviewed by Kurj an I et al. (1992) Annu. Rev. Biochem. 61:1097-1129). In addition to these groups of GPCRs, there are a small number of other proteins which present seven putative hydrophobic segments and appear to be unrelated to GPCRs; however, they have not been shown to couple to G-proteins. Drosophila express a photoreceptor-specific protein bride of sevenless (boss), a seven-transmembrane-segment protein which has been extensively studied and does not show evidence of being a GPCR (Hart et al. (1993) Proc. Natl. Acad. Sci. USA 90:5047-5051). The gene frizzled (fz) in Drosophila is also thought to be a protein with seven transmembrane segments. Like boss, fz has not been shown to couple to G-proteins (Vinson et al., Nature 338:263-264 (1989)).

G proteins represent a family of heterotrimeric proteins composed of α, β and γ subunits, which bind guanine nucleotides. These proteins are usually linked to cell surface receptors, e.g., receptors containing seven transmembrane domains. Following ligand binding to the GPCR, a conformational change is transmitted to the G protein, which causes the α-subunit to exchange a bound GDP molecule for a GTP molecule and to dissociate from the βγ-subunits. The GTP-bound form of the α-subunit typically functions as an effector-modulating moiety, leading to the production of second messengers, such as cyclic AMP (e.g., by activation of adenylate cyclase), diacylglycerol or inositol phosphates. Greater than 20 different types of α-subunits are known in man, which associate with a smaller pool of β and γ subunits. Examples of mammalian G proteins include Gi, Go, Gq, Gs and Gt. G proteins are described extensively in Lodish H. et al. Molecular Cell Biology, (Scientific American Books Inc., New York, N.Y., 1995).

GPCRs are a major target for drug action and development. Accordingly, it is valuable to the field of pharmaceutical development to identify and characterize previously unknown GPCRs.

### SUMMARY OF THE INVENTION

The present invention is based, at least in part, on the discovery of novel G-protein coupled receptor (GPCR) family members, referred to herein as “SLGP” nucleic acid and protein molecules. The SLGP molecules of the present invention are useful as targets for developing modulating agents to regulate a variety of cellular processes. Accordingly, in one aspect, this invention provides isolated nucleic acid molecules encoding SLGP proteins or biologically active portions thereof, as well as nucleic acid fragments suitable as primers or hybridization probes for the detection of SLGP-encoding nucleic acids.

In one embodiment, an SLGP nucleic acid molecule of the invention is at least 40%, 42%, 45%, 50%, 55%, 60%, 65%, 70%, 75%, 80%, 85%, 90%, 95%, 98%, or more homologous to the nucleotide sequence (e.g., to the entire length of the nucleotide sequence) shown in SEQ ID NO:1, SEQ ID NO:3, or the nucleotide sequence of the DNA insert of the plasmid deposited with ATCC as Accession Number ______, or a complement thereof.

In a preferred embodiment, the isolated nucleic acid molecule includes the nucleotide sequence shown SEQ ID NO:1 or 3, or a complement thereof. In another embodiment, the nucleic acid molecule includes SEQ ID NO:3 and nucleotides 1-109 of SEQ ID NO:1. In another embodiment, the nucleic acid molecule includes SEQ ID NO:3 and nucleotides 1298-1537 of SEQ ID NO:1. In another preferred embodiment, the nucleic acid molecule consists of the nucleotide sequence shown in SEQ ID NO:1 or 3. In another preferred embodiment, the nucleic acid molecule includes a fragment of at least 239 nucleotides of the nucleotide sequence of SEQ ID NO:1, SEQ ID NO:3, or a complement thereof.

In another embodiment, an SLGP nucleic acid molecule includes a nucleotide sequence encoding a protein having an amino acid sequence sufficiently homologous to the amino acid sequence of SEQ ID NO:2 or an amino acid sequence encoded by the DNA insert of the plasmid deposited with ATCC as Accession Number ______. In a preferred embodiment, an SLGP nucleic acid molecule includes a nucleotide sequence encoding a protein having an amino acid sequence at least 28%, 30%, 35%, 40%, 45%, 50%, 55%, 60%, 65%, 70%, 75%, 80%, 85%, 90%, 95%, 98%, or more homologous to the amino acid sequence of SEQ ID NO:2 or the amino acid sequence encoded by the DNA insert of the plasmid deposited with ATCC as Accession Number ______.

In another preferred embodiment, an isolated nucleic acid molecule encodes the amino acid sequence of human SLGP. In yet another preferred embodiment, the nucleic acid molecule includes a nucleotide sequence encoding a protein having the amino acid sequence of SEQ ID NO: 2 or the amino acid sequence encoded by the DNA insert of the plasmid deposited with ATCC as Accession Number ______. In yet another preferred embodiment, the nucleic acid molecule is at least 2070 nucleotides in length. In a further preferred embodiment, the nucleic acid molecule is at least 2070 nucleotides in length and encodes a protein having an SLGP activity (as described herein).

Another embodiment of the invention features nucleic acid molecules, preferably SLGP nucleic acid molecules, which specifically detect SLGP nucleic acid molecules relative to nucleic acid molecules encoding non-SLGP proteins. For example, in one embodiment, such a nucleic acid molecule is at least 1930, 1900-2000, 1700-2200, 1500-2400, 1300-2600, 1100-2800 or more nucleotides in length and hybridizes under stringent conditions to a nucleic acid molecule comprising the nucleotide sequence shown in SEQ ID NO:1, the nucleotide sequence of the DNA insert of the plasmid deposited with ATCC as Accession Number ______, or a complement thereof. In preferred embodiments, the nucleic acid molecules are at least 15 (e.g., contiguous) nucleotides in length and hybridize under stringent conditions to nucleotides 1-569 and 1058-2987 of SEQ ID NO:1. In other preferred embodiments, the nucleic acid molecules comprise nucleotides 1-569 and 1058-2987 of SEQ ID NO:1.

In other preferred embodiments, the nucleic acid molecule encodes a naturally occurring allelic variant of a polypeptide comprising the amino acid sequence of SEQ ID NO:2 or an amino acid sequence encoded by the DNA insert of the plasmid deposited with ATCC as Accession Number ______, wherein the nucleic acid molecule hybridizes to a nucleic acid molecule comprising SEQ ID NO:1 or SEQ ID NO:3 under stringent conditions.

Another embodiment of the invention provides an isolated nucleic acid molecule which is antisense to an SLGP nucleic acid molecule, e.g., the coding strand of an SLGP nucleic acid molecule.

Another aspect of the invention provides a vector comprising an SLGP nucleic acid molecule. In certain embodiments, the vector is a recombinant expression vector. In another embodiment, the invention provides a host cell containing a vector of the invention. The invention also provides a method for producing a protein, preferably an SLGP protein, by culturing in a suitable medium, a host cell, e.g., a mammalian host cell such as a non-human mammalian cell, of the invention containing a recombinant expression vector, such that the protein is produced.

Another aspect of this invention features isolated or recombinant SLGP proteins and polypeptides. In one embodiment, the isolated protein, preferably an SLGP protein, includes at least one transmembrane domain. In a preferred embodiment, the isolated protein, preferably an SLGP protein, includes seven transmembrane domains. In a preferred embodiment, the protein, preferably an SLGP protein, includes at least one transmembrane domain and has an amino acid sequence at least about 28%, 30%, 35%, 40%, 45%, 50%, 55%, 60%, 65%, 70%, 75%, 80%, 85%, 90%, 95%, 98% or more homologous to the amino acid sequence of SEQ ID NO:2 or the amino acid sequence encoded by the DNA insert of the plasmid deposited with ATCC as Accession Number ______. In another preferred embodiment, the protein, preferably an SLGP protein, includes at least one transmembrane domain and plays a role in the mobilization of intracellular molecules that participate in a signal transduction pathway, e.g., phosphatidylinositol 4,5-bisphosphate (PIP₂), inositol 1,4,5-triphosphate (IP₃), or adenylate cyclase; the production or secretion of molecules; alteration in the structure of a cellular component; cell proliferation, e.g., synthesis of DNA; cell migration; cell differentiation; or cell survival. In another preferred embodiment, the protein, preferably an SLGP protein, includes at least one transmembrane domain and plays a role in the signal trandsuction cascade associated with the establishment or development of an inflammatory process (e.g., leukocyte activation). In yet another preferred embodiment, the protein, preferably an SLGP protein, includes at least one transmembrane domain and is encoded by a nucleic acid molecule having a nucleotide sequence which hybridizes under stringent hybridization conditions to a nucleic acid molecule comprising the nucleotide sequence of SEQ ID NO:1 or SEQ ID NO:3.

In another embodiment, the invention features fragments of the protein having the amino acid sequence of SEQ ID NO:2, wherein the fragment comprises at least 15 amino acids (e.g., contiguous amino acids) of the amino acid sequence of SEQ ID NO:2 or an amino acid sequence encoded by the DNA insert of the plasmid deposited with the ATCC as Accession Number ______. In another embodiment, the protein, preferably an SLGP protein, has the amino acid sequence of SEQ ID NO:2.

In another embodiment, the invention features an isolated protein, preferably an SLGP protein, which is encoded by a nucleic acid molecule consisting of a nucleotide sequence at least about 40%, 42%, 45%, 50%, 55%, 60%, 65%, 70%, 75%, 80%, 85%, 90%, 95%, 98% or more homologous to a nucleotide sequence of SEQ ID NO:1, SEQ ID NO:3, or a complement thereof. This invention further features an isolated protein, preferably an SLGP protein, which is encoded by a nucleic acid molecule consisting of a nucleotide sequence which hybridizes under stringent hybridization conditions to a nucleic acid molecule comprising the nucleotide sequence of SEQ ID NO:1, SEQ ID NO:3, or a complement thereof.

The proteins of the present invention or portions thereof, e.g., biologically active portions thereof, can be operatively linked to a non-SLGP polypeptide (e.g., heterologous amino acid sequences) to form fusion proteins. The invention further features antibodies, such as monoclonal or polyclonal antibodies, that specifically bind proteins of the invention, preferably SLGP proteins. In addition, the SLGP proteins or biologically active portions thereof can be incorporated into pharmaceutical compositions, which optionally include pharmaceutically acceptable carriers.

In another aspect, the present invention provides a method for detecting the presence of an SLGP nucleic acid molecule, protein or polypeptide in a biological sample by contacting the biological sample with an agent capable of detecting an SLGP nucleic acid molecule, protein or polypeptide such that the presence of an SLGP nucleic acid molecule, protein or polypeptide is detected in the biological sample.

In another aspect, the present invention provides a method for detecting the presence of SLGP activity in a biological sample by contacting the biological sample with an agent capable of detecting an indicator of SLGP activity such that the presence of SLGP activity is detected in the biological sample.

In another aspect, the invention provides a method for modulating SLGP activity comprising contacting a cell capable of expressing SLGP with an agent that modulates SLGP activity such that SLGP activity in the cell is modulated. In one embodiment, the agent inhibits SLGP activity. In another embodiment, the agent stimulates SLGP activity. In one embodiment, the agent is an antibody that specifically binds to an SLGP protein. In another embodiment, the agent modulates expression of SLGP by modulating transcription of an SLGP gene or translation of an SLGP mRNA. In yet another embodiment, the agent is a nucleic acid molecule having a nucleotide sequence that is antisense to the coding strand of an SLGP mRNA or an SLGP gene.

In one embodiment, the methods of the present invention are used to treat a subject having a disorder characterized by aberrant SLGP protein or nucleic acid expression or activity by administering an agent which is an SLGP modulator to the subject. In one embodiment, the SLGP modulator is an SLGP protein. In another embodiment the SLGP modulator is an SLGP nucleic acid molecule. In yet another embodiment, the SLGP modulator is a peptide, peptidomimetic, or other small molecule. In a preferred embodiment, the disorder characterized by aberrant SLGP protein or nucleic acid expression is a proliferative disorder, a differentiative or developmental disorder, or a hematopoietic disorder.

The present invention also provides a diagnostic assay for identifying the presence or absence of a genetic alteration characterized by at least one of (i) aberrant modification or mutation of a gene encoding an SLGP protein; (ii) mis-regulation of the gene; and (iii) aberrant post-translational modification of an SLGP protein, wherein a wild-type form of the gene encodes an protein with an SLGP activity.

In another aspect the invention provides a method for identifying a compound that binds to or modulates the activity of an SLGP protein, by providing an indicator composition comprising an SLGP protein having SLGP activity, contacting the indicator composition with a test compound, and determining the effect of the test compound on SLGP activity in the indicator composition to identify a compound that modulates the activity of an SLGP protein.

Other features and advantages of the invention will be apparent from the following detailed description and claims.

### BRIEF DESCRIPTION OF THE DRAWINGS

FIG. 1 depicts the cDNA sequence of human SLGP. The nucleotide sequence corresponds to nucleic acids 1 to 2987 of SEQ ID NO:1.

FIG. 2 depicts the predicted amino acid sequence of human SLGP. The amino acid sequence corresponds to amino acids 1 to 690 of SEQ ID NO:2.

FIG. 3 depicts the coding region of the cDNA sequence of human SLGP. The nucleotide sequence corresponds to amino acids 1 to 2070 of SEQ ID NO:3.

FIG. 4 depicts an alignment of the amino acid sequences of human SLGP (SEQ ID NO:2) and human CD 97 (Accession No. U76764, SEQ ID NO:X). This alignment were generated utilizing the ALIGN program with the following parameter setting: PAM120, gap penalties: -12/-4 (Myers, E. and Miller, W. (1988) “Optimal Alignments in Linear Space” CABIOS 4:11-17).

FIG. 5 depicts an alignment of the nucleotide sequences of human SLGP (SEQ ID NO:2) and human CD 97 (Accession No. U76764, SEQ ID NO:X). This alignment were generated utilizing the ALIGN program with the following parameter setting: PAM120, gap penalties: -12/-4 (Myers, E. and Miller, W. (1988) “Optimal Alignments in Linear Space” CABIOS 4:11-17).

### DETAILED DESCRIPTION OF THE INVENTION

The present invention is based, at least in part, on the discovery of novel G-protein coupled receptor (GPCR) family members, referred to herein as SLGP protein and nucleic acid molecules. These molecules comprise a family of molecules having certain conserved structural and functional features. The term “family” when referring to the protein and nucleic acid molecules of the invention is intended to mean two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin, as well as other, distinct proteins of human origin or alternatively, can contain homologues of non-human origin. Members of a family may also have common functional characteristics.

For example, the family of G protein-coupled receptors (GPCRs), to which the SLGP proteins of the present invention bear significant homology, comprise an N-terminal extracellular domain, seven transmembrane domains (also referred to as membrane-spanning domains), three extracellular domains (also referred to as extracellular loops), three cytoplasmic domains (also referred to as cytoplasmic loops), and a C-terminal cytoplasmic domain (also referred to as a cytoplasmic tail). Members of the SLGP family also share certain conserved amino acid residues, some of which have been determined to be critical to receptor function and/or G protein signaling. For example, GPCRs usually contain the following features: a conserved asparagine residue in the first transmembrane domain; a cysteine residue in the first extracellular loop which is believed to form a disulfide bond with a conserved cysteine residue in the second extracellular loop; a conserved leucine and aspartate residue in the second transmembrane domain; an aspartate-arginine-tyrosine motif (DRY motif) at the interface of the third transmembrane domain and the second cytoplasmic loop of which the arginine residue is almost invariant (members of the rhodopsin subfamily of GPCRs comprise a histidine-arginine-methionine motif (HRM motif) as compared to a DRY motif); a conserved tryptophan and proline residue in the fourth transmembrane domain; a conserved phenylalanine residue which is commonly found as part of the motif FXXCXXP; and a conserved leucine residue in the seventh transmembrane domain which is commonly found as part of the motif DPXXY or NPXXY. Table I depicts an alignment of the transmembrane domain of 5 GPCRs. The conserved residues described herein are indicated by asterices. An alignment of the transmembrane domains of 44 representative GPCRs can be found at http://mgdkkl.nidll.nih.gov:8000/extended.html.

TABLE I
ALIGNMENT OF:

| | | | |
|---|---|---|---|
| thrombin | (6.) | human | P25116 |
| rhodopsin | (19.) | human | P08100 |
| m1ACh | (21.) | rat | P08482 |
| IL-8A | (30.) | human | P25024 |
| octopamine | (40.) | Drosophila melanogaster | P22270 |

| | | | |
|---|---|---|---|
| TM1 | | | |
| | | * | |
| 6. | 102 | TLFVPSVYTGVFVVSLPLNIMAIVVFILKMK | 132 |
| 19. | 37 | FSMLAAYMFLLIVLGFPINFLTLYVTVQHKK | 67 |
| 21. | 25 | VAFIGITTGLLSLATVTGNLLVLISFKVNTE | 55 |
| 30. | 39 | KYVVIIAYALVFLLSLLGNSLVMLVILYSRV | 69 |
| 40. | 109 | ALLTALVLSVIIVLTIIGNILVILSVFTYKP | 139 |
| | | \| | |
| | | 1111111111111111111111111111111 | |
| | | 3333333344444444445555555555666 | |
| | | 2345678901234567890123456789012 | |
| TM2 | | | |
| | | * * | |
| 6. | 138 | VVYMLHLATADVLFVSVLPFKISYYFSG | 165 |
| 19. | 73 | NYILLNLAVADLFMVLGGFTSTLYTSLH | 100 |
| 21. | 61 | NYFLLSLACADLIIGTFSMNLYTTYLLM | 88 |
| 30. | 75 | DVYLLNLALADLLFALTLPIWAASKVNG | 102 |
| 40. | 145 | NFFIVSLAVADLTVALLVLPFNVAYSIL | 172 |
| | | \| | |
| | | 2222222222222222222222222222 | |
| | | 4444444444555555555566666666 | |
| | | 0123456789012345678901234567 | |
| TM3 | | | |
| | | * | |
| 6. | 176 | RFVTAAFYCNMYASILLMTVISIDR | 200 |
| 19. | 111 | NLEGFFATLGGEIALWSLVVLAIER | 135 |
| 21. | 99 | DLWLALDYVASNASVMNLLLISFDR | 123 |
| 30. | 111 | KVVSLLKEVNFYSGILLLACISVDR | 135 |
| 40. | 183 | KLWLTCDVLCCTSSILNLCAIALDR | 207 |
| | | \| | |
| | | 3333333333333333333333333 | |
| | | 2222333333333344444444445 | |
| | | 6789012345678901234567890 | |
| TM4 | | | |
| | | * * | |
| 6. | 215 | TLGRASFTCLAIWALAIAGVVPLVLKE | 241 |
| 19. | 149 | GENHAIMGVAFTWVMALACAAPPLAGW | 175 |
| 21. | 138 | TPRPAALMIGLAWLVSFVLWAPAILFW | 164 |
| 30. | 149 | KRHLVKFVCLGCWGLSMNLSLPFFLFR | 175 |
| 40. | 222 | TVGRVLLLISGVWLLSLLISSPPLIGW | 248 |
| | | \| | |
| | | 444444444444444444444444444 | |
| | | 334444444444555555555566666 | |
| | | 890123456789012345678901234 | |
| TM5 | | | |
| | | * * * | |
| 6. | 268 | AYYFSAFSAVFFFVPLIISTVCYVSIIRC | 296 |
| 19. | 201 | ESFVIYMFVVHFTIPMIIIFFCYGQLVFT | 229 |
| 21. | 186 | PIITFGTAMAAFYLPVTVMCTLYWRIYRE | 214 |
| 30. | 200 | MVLRILPHTFGFIVPLFVMLFCYGFTLRT | 228 |
| 40. | 267 | RGYVIYSSLGSFFIPLAIMTIVYIEIFVA | 295 |
| | | \| | |
| | | 55555555555555555555555555555 | |
| | | 33334444444444555555555566666 | |
| | | 67890123456789012345678901234 | |
| TM6 | | | |
| | | * * * | |
| 6. | 313 | FLSAAVFCIFIICFGPTNVLLIAHYSFL | 340 |
| 19. | 252 | RMVIIMVIAFLICWVPYASVAFYIFTHQ | 279 |
| 21. | 365 | RTLSAILLAFILTWTPYNIMVLVSTFCK | 397 |
| 30. | 242 | RVIFAVVLIFLLCWLPYNLVLLADTLMR | 269 |
| 40. | 529 | RTLGIIMGVFVICWLPFFLMYVILPFCQ | 556 |
| | | \| | |
| | | 6666666666666666666666666666 | |
| | | 3333344444444445555555556666 | |
| | | 5678901234567890123456789012 | |
| TM7 | | | |
| | | ** * | |
| 6. | 347 | EAAYFAYLLCVCVSSISSCIDPLIYYYASSECQ | 379 |
| 19. | 282 | NFGPIFMTIPAFFAKSAAIYNPVIYIMMNKQFR | 314 |
| 21. | 394 | CVPETLWELGYWLCYVNSTVNPMCYALCNKAFR | 426 |
| 30. | 281 | NNIGRALDATEILGFLHSCLNPIIYAFIGQNFR | 313 |
| 40. | 559 | CPTNKFKNFITWLGYINSGLNPVIYTIFNLDYR | 591 |
| | | \| | |
| | | 777777777777777777777777777777777 | |
| | | 233333333334444444444555555555566 | |
| | | 901234567890123456789012345678901 | |

The amino acid sequences of thrombin (Accession No. P25116), rhodopsin (Accession No. P08100), ml ACh (Accession No. P08482), IL-8A (Accession No. P25024), octopamine (Accession No. P22270), can be found as SEQ ID NO:XX, SEQ ID NO:XX, SEQ ID NO:XX, SEQ ID NO:XX, and SEQ ID NO:XX, respectively. Accordingly, GPCR-like proteins such as the SLGP proteins of the present invention contain a siginificant number of structural characteristics of the GPCR family. For instance, the SLGPs of the present invention contain conserved cysteines found in the first 2 extracellular loops (prior to the third and fifth transmembrane domains) of most GPCRs (cys490 and cys602 of SEQ ID NO:2). A highly conserved asparagine residue is present (asn125 in SEQ ID NO:2). SLGP proteins contains a highly conserved leucine (leu154 of SEQ ID NO:2). The two cysteine residues are believed to form a disulfide bond that stabilizes the functional protein structure. A highly conserved asparagine and arginine in the fourth transmembrane domain of the SLGP proteins is present (asp158 and arg218 of SEQ ID NO:2). The third cytoplasmic loop contains 18 amino acid residues and is thus the longest cytoplasmic loop of the three, characteristic of G protein coupled receptors. Moreover, a highly conserved proline is present (pro307 of SEQ ID NO:2). Proline residues in the fourth, fifth, sixth, and seventh transmembrane domains are thought to introduce kinks in the alpha-helices and may be important in the formation of the ligand binding pocket. Moreover, a conserved tyrosine is present in the seventh transmembrane domain of SLGP-2 (tyr646 of SEQ ID NO:2).

In one embodiment, the SLGP proteins of the present invention are proteins having an amino acid sequence of about 450-850, preferably about 500-800, more preferably about 550-750, more preferably about 600-700, or about 650-690 amino acids in length. In one embodiment, the SLGP proteins of the present invention contain at least one, two, three, four, five, six, or preferably, seven transmembrane domains. As used herein, the term “transmembrane domain” includes an amino acid sequence of about 15-40 amino acid residues in length, more preferably, about 15-30 amino acid residues in length, and most preferably about 18-25 amino acid residues in length, which spans the plasma membrane. Transmembrane domains are rich in hydrophobic residues, and typically have an α-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta W. N. et al, (1996) Annual Rev. Neuronsci. 19: 235-63, the contents of which are incorporated herein by reference. In a preferred embodiment, a SLGP protein of the present invention has more than one transmembrane domain, preferably 2, 3, 4, 5, 6, or 7 transmembrane domains. For example, transmembrane domains can be found at about amino acids 433-452, 465-481, 500-524, 533-553, 570-594, 619-635, and 642-666 of SEQ ID NO:2. In a particularly preferred embodiment, a SLGP protein of the present invention has 7 transmembrane domains.

In another embodiment, a SLGP is identified based on the presence of at least one cytoplasmic loop, also referred to herein as a cytoplasmic domain. In another embodiment, a SLGP is identified based on the presence of at least one extracellular loop. As defined herein, the term “loop” includes an amino acid sequence having a length of at least about 4, preferably about 5-10, preferably about 10-20, and more preferably about 20-30, 30-40, 40-50, 50-60, 60-70, 70-80, 80-90, 90-100, or 100-150 amino acid residues, and has an amino acid sequence that connects two transmembrane domains within a protein or polypeptide. Accordingly, the N-terminal amino acid of a loop is adjacent to a C-terminal amino acid of a transmembrane domain in a naturally-occurring SLGP or SLGP-like molecule, and the C-terminal amino acid of a loop is adjacent to an N-terminal amino acid of a transmembrane domain in a naturally-occurring SLGP or SLGP-like molecule.

As used herein, a “cytoplasmic loop” includes an amino acid sequence located within a cell or within the cytoplasm of a cell. For example, a cytoplasmic loop is found at about amino acids 453-464, 525-532, and 595-618 of SEQ ID NO:2. Also as used herein, an “extracellular loop” includes an amino acid sequence located outside of a cell, or extracellularly. For example, an extracellular loop can be found at about amino acid residues 482-499, 554-569, and 636-641 of SEQ ID NO:2.

In another embodiment of the invention, a SLGP is identified based on the presence of a “C-terminal cytoplasmic domain”, also referred to herein as a C-terminal cytoplasmic tail, in the sequence of the protein. As used herein, a “C-terminal cytoplasmic domain” includes an amino acid sequence having a length of at least about 10, preferably about 10-25, more preferably about 25-50, more preferably about 50-75, even more preferably about 75-100, 100-150, 150-200, 200-250, 250-300, 300-400, 400-500, or 500-600 amino acid resudues and is located within a cell or within the cytoplasm of a cell. Accordingly, the N-terminal amino acid residue of a “C-terminal cytoplasmic domain” is adjacent to a C-terminal amino acid residue of a transmembrane domain in a naturally-occurring SLGP or SLGP-like protein. For example, a C-terminal cytoplasmic domain is found at about amino acid residues 595-618 of SEQ ID NO:2.

In another embodiment, a SLGP is identified based on the presence of an “N-terminal extracellular domain”, also referred to herein as an N-terminal extracellular loop in the amino acid sequence of the protein. As used herein, an “N-terminal extracellular domain” includes an amino acid sequence having about 1-500, preferably about 1-400, more preferably about 1-300, more preferably about 1-200, even more preferably about 1-100, and even more preferably about 1-50, 1-25, or 1-10 amino acid residues in length and is located outside of a cell or extracellularly. The C-terminal amino acid residue of a “N-terminal extracellular domain” is adjacent to an N-terminal amino acid residue of a transmembrane domain in a naturally-occurring SLGP or SLGP-like protein. For example, an N-terminal extracellular domain is found at about amino acid residues 21-481 of SEQ ID NO:2.

Accordingly in one embodiment of the invention, an SLGP includes at least one, preferably 6 or 7, transmembrane domains and and/or at least one cytoplasmic loop, and/or at least one extracellular loop. In another embodiment, the SLGP further includes an N-terminal extracellular domain and/or a C-terminal cytoplasmic domain. In another embodiment, the SLGP can include six transmembrane domains, three cytoplasmic loops, and two extracellular loops, or can include six transmembrane domains, three extracellular loops, and 2 cytoplasmic loops. The former embodiment can further include an N-terminal extracellular domain. The latter embodiment can further include a C-terminal cytoplasmic domain. In another embodiment, the SLGP can include seven transmembrane domains, three cytoplasmic loops, and three extracellular loops and can further include an N-terminal extracellular domain or a C-terminal cytoplasmic domain.

In another embodiment, a SLGP is identified based on the presence of at least one “7 transmembrane receptor profile”, also referred to as a “Secretin family sequence profile”, in the protein or corresponding nucleic acid molecule. As used herein, the term “7 transmembrane receptor profile” includes an amino acid sequence having at least about 50-350, preferably about 100-300, more preferably about 150-275 amino acid residues, or at least about 200-258 amino acids in length and having a bit score for the alignment of the sequence to the 7tm_{—}1 family Hidden Markov Model (HMM) of at least 20, preferably 20-30, more preferably 30-40, more preferably 40-50, or 50-75 or greater. The 7tm_{—}1 family HMM has been assigned the PFAM Accession PF000001 (http://genome.wustl.edu/Pfam/WWWdata/7tm_{—}1.html).

To identify the presence of a 7 transmembrane receptor profile in a SLGP, the amino acid sequence of the protein is searched against a database of HMMs (e.g., the Pfam database, release 2.1) using the default parameters (http://www.sanger.ac.uk/Software/Pfam/HMM_search). For example, the hmmsf program, which is available as part of the HMMER package of search programs, is a family specific default program for PF00002 and score of 15 is the default threshold score for determining a hit. For example, a search using the amino acid sequence of SEQ ID NO:2 was performed against the HMM database resulting in the identification of a 7 TM receptor profile in the amino acid sequence of SEQ ID NO:2. The results of the search are set forth below.

Score: 56.37 Seq: 421 678 Model: 75 348

| | | | |
|---|---|---|---|
| | | *ksYYyvvYiIYTVGYSMSiaaLlvAMfIFcfFRrLHCtRNYIHMNMFms | |
| | | +++Y+++ I +G +S++ L + +F F FF + TR +IH+N+ S | |
| SLGP | 421 | IKDYNILTRITQLGIIISLICLAICIFTFWFFSEIQSTRTTIHKNLCCS | 469 |
| | | FILRaisWFIkDWvlyWmYsndeltwHCwMsivwCRivMfFMQYMMMtNY | |
| | | L A +F++ +N +C I +Y+ ++ + | |
| SLGP | 470 | LFL-AELVFLVGINT---NTNKL----------FCSIIAGLLHYFFLAAF | 505 |
| | | FWMLvEGvYLHTLIvMtFFsERqYFWWYylIGWGfPlVFitiWvItRcyY | |
| | | WM +EG+ L+ +V + + +Y++G +P+V ++ + + Y | |
| SLGP | 506 | AWMCIEGIHLYLIVVGVIYNKGFLHKNFYIFGYLSPAVVVGFSAALGYRY | 555 |
| | | ENt..nCWDmNDnMwyWWIIrgPIMlsIvVNFFFFINIIRILMtKLRepq | |
| | | + T CW++++N ++ W +GP L I+ N++ F II+ + + | |
| SLGP | 556 | YGTTKVCWLSTEN-NFIWSFIGPACLIILGNLLAFGVIIYKVFRHTAGLK | 604 |
| | | MgEndMqqYWRlvKSTLlLIPLFGIHYMVFaWrPdNhwlwqIYMYFElsl | |
| | | + + + L L+ + +F + +++ Y+ + | |
| SLGP | 605 | PEVSCF--ENIRSCARGALALLLLGTTWIFGGLHVV-HASVVTAYLFTVS | 651 |
| | | iSFQGFFVAiIYCFcNhEVQmEIRRrW* | |
| | | + FQG+F + C + + Q+E R | |
| SLGP | 652 | NAFQGMFIFLFLCVLSRKIQEEYYRLF 678 | |

Accordingly, in one embodiment of the invention, a SLGP protein is a human SLGP protein having a 7 transmembrane receptor profile at about amino acids 433-666 of SEQ ID NO:2. Such a 7 transmembrane receptor profile has the amino acid sequence:

(SEQ ID NO:XX)

IKDYNILTRITQLGIIISLICLAICIFTFWFFSEIQSTRTTIHKNLCCSL
FLAELVFLVGINTNTNKLFCSIIAGLLHYFFLAAFAWMCIEGIHLYLIVV
GVIYNKGFLHKNFYIFGYLSPAVVVGFSAALGYRYYGTTKVCWLSTENNF
IWSFIGPACLIILGNLLAFGVIIYKVFRHTAGLKPEVSCFENIRSCARGA
LALLLLGTTWIFGGLHVVHASVVTAYLFTVSNAFQGMFIFLFLCVLSRKI
QEEYYRLF

Accordingly, SLGP proteins having at least 20-30%, 30-49%, 40-50%, 50-60% homology, preferably about 60-70%, more preferably about 70-80%, or about 80-90% homology with the 7 transmembrane receptor profile of human SLGP (e.g., SEQ ID NO:2) are within the scope of the invention.

In another embodiment, a SLGP is identified based on the presence of a “EGF-like domain” in the protein or corresponding nucleic acid molecule. As used herein, the term “EGF-like domain” includes a protein domain having an amino acid sequence of about 55-90, preferably about 60-85, more preferably about 65-80 amino acid residues, or about 70-79 amino acids and having a bit score for the alignment of the sequence to the EGF-like domain (HMM) of at least 6, preferably 7-10, more preferably 10-30, more preferably 30-50, even more preferably 50-75, 75-100, 100-200 or greater. The EGF-like domain HMM has been assigned the PFAM Accession PF00008 (http://genome.wustl.edu/Pfam/WWWdata/EGF.html). Preferably, one or more cysteine residues in the EGF-like domain are conserved among SLGP family members or other proteins containing EGF-like domains (i.e., located in the same or similar position as the cysteine residues in other SLGP family members or other proteins containing EGF-like domains). In a preferred embodiment, an “EGF-like domain” has the consensus sequence X(4)-C-X(0,48)-C-X(3,12)-C-X(1,70)-C-X(1,6)-C-X(2)-G-a-X(0,21)-G-X(2)-C-X, C=conserved cysteine involved in a disulfide bond, G=often conserved glycine, a=often conserved aromatic acid, X=any residue; corresponding to SEQ ID NO:XX. In another referred embodiment, an “EGF-like domain” has the consensus sequence C-X-C-X(5)-G-X(2)-C, the 3 C's are involved in disulfide bonds; corresponding to SEQ ID NO:XX. In another preferred embodiment, an “EGF-like domain” has the consensus sequence C-X-C-X(2)-[GP]-[FYW]-X(4,8)-C, the three C's are involved in disulfide bonds; corresponding to SEQ ID NO:XX.

To identify the presence of a EGF-like domain in a SLGP protein, make the determination that a protein of interest has a particular profile, the amino acid sequence of the protein is searched against a database of HMMs (e.g., the Pfam database, release 2.1) using the default parameters (http://www.sanger.ac.uk/Software/Pfam/HMM_search). For example, the hmmsf program, which is available as part of the HMMER package of search programs, is a family specific default program for PF00008 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3)405-420 and a detailed description of HMMs can be found, for example, in Gribskov et al.(1990) Meth. Enzymol. 183:146-159; Gribskov et al.(1987) Proc. Natl. Acad. Sci. USA 84:4355-4358; Krogh et al.(1994) J. Mol. Biol. 235:1501-1531; and Stultz et al.(1993) Protein Sci. 2:305-314, the contents of which are incorporated herein by reference. A search was performed against the HMM database resulting in the identification of a EGF-like domain in the amino acid sequence of SEQ ID NO:2. The results of the search are set forth below.

Score: 6.16 Seq: 22 53 Model: 1 34

| | | | |
|---|---|---|---|
| | | *CnpNPCmNgGtCvNtp.mYtCiCpeGYmyYtGrrC* | |
| | | C+ +PC+ +++C+ C C +G ++G | |
| SLGP | 22 | CTKTPCLPNAKCEIRNGIEACYCNMG---FSGNGV | 53 |

Score: 18.87 Seg: 62 100 Model: 1 34

| | | | |
|---|---|---|---|
| | | *CnpN..PCmNgGtCvNtp.mYtCiCpeGYm.y.YtGrrC* | |
| | | C ++ C +++ C+NT+ +Y+C C +G++ + + R+ | |
| SLGP | 62 | CGNLTQSCGENANCTNTEGSYYCMCVPGFRSSSNQDRFI | 100 |

All amino acids are described using universal single letter abbreviations according to these motifs.

Such a EGF-like domain has the amino acid sequence:

| | |
|---|---|
| CTKTPCLPNAKCEIRNGIEACYCNMGFSGNGV | (SEQ ID NO:XXX) |
| CGNLTQSCGENANCTNTEGSYYCMCVPGFRSSSN | (SEQ ID NO:XXX) |

QDRFI.

Accordingly, SLGP proteins having at least 50-60% homology, preferably about 60-70%, more preferably about 70-80%, or about 80-90% homology with a EGF-like domain of human SLGP (e.g., SEQ ID NO:XXX) are within the scope of the invention.

In another embodiment, a SLGP is identified based on the presence of a “NADH-ubiquinone/plastoquinone oxidoreductase chain 4L domain” in the protein or corresponding nucleic acid molecule. As used herein, the term “NADH-ubiquinone/plastoquinone oxidoreductase chain 4L domain” includes a protein domain having an amino acid sequence of about 25-55, preferably about 30-50, more preferably about 35-45 amino acid residues, or about 40-43 amino acids and having a bit score for the alignment of the sequence to the NADH-ubiquinone/plastoquinone oxidoreductase chain 4L domain (HMM) of at least 6, preferably 7-10, more preferably 10-30, more preferably 30-50, even more preferably 50-75, 75-100, 100-200 or greater. The NADH ubiquinone/plastoquinone oxidoreductase chain 4L domain HMM has been assigned the PFAM Accession PF00008 (http://genome.wustl.edu/Pfam/WWWdata/XXX.html).

To identify the presence of a NADH-ubiquinone/plastoquinone oxidoreductase chain 4L domain in a SLGP protein, make the determination that a protein of interest has a particular profile, the amino acid sequence of the protein is searched against a database of HMMs (e.g., the Pfam database, release 2.1) using the default parameters (http://www.sanger.ac.uk/Software/Pfam/HMM_search). For example, the hmmsf program, which is available as part of the HMMER package of search programs, is a family specific default program for PF00420 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3)405-420 and a detailed description of HMMs can be found, for example, in Gribskov et al.(1990) Meth. Enzymol. 183:146-159; Gribskov et al.(1987) Proc. Natl. Acad. Sci. USA 84:4355-4358; Krogh et al.(1994) J. Mol. Biol. 235:1501-1531; and Stultz et al.(1993) Protein Sci. 2:305-314, the contents of which are incorporated herein by reference. A search was performed against the HMM database resulting in the identification of a NADH-ubiquinone/plastoquinone oxidoreductase chain 4L domain in the amino acid sequence of SEQ ID NO:2. The results of the search are set forth below.

Score: 6.77 Seq: 475 517 Model: 1 43

| | | | |
|---|---|---|---|
| | | *MMMMthYHFiIMIaFmmGIMGIlMNRsHmMSMLMCLEmMMLSl* | |
| | | ++ + ++ +F+ I G+L + ++ MC+E++ L L | |
| SLGP | 475 | LVFLVGINTNTNKLFCSIIAGLLHYFFLAAFAWMCIEGIHLYL | 517 |

All amino acids are described using universal single letter abbreviations according to these motifs.

Such a NADH-ubiquinone/plastoquinone oxidoreductase chain 4L domain has the amino acid sequence:

LVFLVGINTNTNKLFCSIIAGLLHYFFLAAFAWMCIEGIHLYL(S EQ ID NO:XX).

Accordingly, SLGP proteins having at least 50-60% homology, preferably about 60-70%, more preferably about 70-80%, or about 80-90% homology with a NADH-ubiquinone/plastoquinone oxidoreductase chain 4L domain of human SLGP (e.g., SEQ ID NO:XXX) are within the scope of the invention.

In another embodiment, a SLGP is identified based on the presence of a “signal sequence” in the protein or corresponding nucleic acid molecule. For example, a signal sequence contains at least about 5-35 amino acid residues, preferably about 10-30 amino acid residues, and more preferably about 15-25 amino acid residues, and has at least about 40-70%, preferably about 50-65%, and more preferably about 55-60% hydrophobic amino acid residues (e.g., Alanine, Valine, Leucine, Isoleucine, Phenylalanine, Tyrosine, Tryptophan, or Proline). Such a “signal sequence”, also referred to in the art as a “signal peptide”, serves to direct a protein containing such a sequence to a lipid bilayer. For example, in one embodiment, a SLGP protein contains a signal sequence of about amino acids 1-20 of SEQ ID NO:2.

In another embodiment, a SLGP protein includes at least a EGF-like domain. In another embodiment, a SLGP protein includes at least an NADH-ubiquinone/plastoquinone oxidoreductase chain 4L domain. In another embodiment, a SLGP protein includes at least a 7 transmembrane receptor profile. In another embodiment, a SLGP protein includes at least a signal sequence. In another embodiment, a SLGP protein includes a EGF-like domain, and an NADH-ubiquinone/plastoquinone oxidoreductase chain 4L domain. In another embodiment, a SLGP protein includes a EGF-like domain and a 7 transmembrane receptor profile. In another embodiment, a SLGP protein includes a EGF-like domain and a signal sequence. In another embodiment, a SLGP protein includes a EGF-like domain, and an NADH-ubiquinone/plastoquinone oxidoreductase chain 4L domain, and a 7 transmembrane receptor profile. In another embodiment, a SLGP protein includes a EGF-like domain, and an NADH-ubiquinone/plastoquinone oxidoreductase chain 4L domain, and a 7 transmembrane receptor profile a signal sequence.

In another embodiment, a SLGP protein includes an NADH-ubiquinone/plastoquinone oxidoreductase chain 4L domain and a 7 transmembrane receptor profile. In another embodiment, a SLGP protein includes an NADH-ubiquinone/plastoquinone oxidoreductase chain 4L domain and a 7 transmembrane receptor profile, and a signal sequence. In another embodiment, a SLGP protein includes an NADH-ubiquinone/plastoquinone oxidoreductase chain 4L domain and a signal sequence.

In another embodiment, a SLGP protein is human SLGP which includes a EGF-like domain having about amino acids 22-100 of SEQ ID NO:2. In another embodiment, a SLGP protein is human SLGP which includes an NADH-ubiquinone/plastoquinone oxidoreductase chain 4L domain having about amino acids 475-517 of SEQ ID NO:2. In another embodiment, a SLGP protein is human SLGP which includes a 7 transmembrane receptor profile having about amino acids 421-678 of SEQ ID NO:2. In another embodiment, a SLGP protein is human SLGP which includes a signal sequence having about amino acids 1-20 of SEQ ID NO:2.

In yet another embodiment, a SLGP protein is human SLGP which includes a a EGF-like domain having about amino acids 22-100 of SEQ ID NO:2, an NADH-ubiquinone/plastoquinone oxidoreductase chain 4L domain having about amino acids 475-517 of SEQ ID NO:2, a 7 transmembrane receptor profile having about amino acids 421-678 of SEQ ID NO:2, and a signal sequence having about amino acids 1-20 of SEQ ID NO:2.

The SLGP protein is a GPCR that participates in signaling pathways within cells, e.g., signaling pathways involved in proliferation or differentiation. As used herein, a signaling pathway refers to the modulation (e.g., the stimulation or inhibition) of a cellular function/activity upon the binding of a ligand to the GPCR (SLGP protein). Examples of such functions include mobilization of intracellular molecules that participate in a signal transduction pathway, e.g., phosphatidylinositol 4,5-bisphosphate (PIP₂), inositol 1,4,5-triphosphate (IP₃) or adenylate cyclase; polarization of the plasma membrane; production or secretion of molecules; alteration in the structure of a cellular component; cell proliferation, e.g., synthesis of DNA; cell migration; cell differentiation; and cell survival. Since the SLGP protein is expressed substantially in white blood cells and neuronal tissues, examples of cells participating in an SLGP signaling pathway include leukocytes and neurons.

Depending on the type of cell, the response mediated by the SLGP protein/ligand binding may be different. For example, in some cells, binding of a ligand to an SLGP protein may stimulate an activity such as leukocyte activation and alpha-Latrotoxin induced exocytosis of small synaptic vesicles in neurons, and the like through phosphatidylinositol or cyclic AMP metabolism and turnover. Regardless of the cellular activity modulated by SLGP, it is universal that as a GPCR, the SLGP protein interacts with a “G protein” to produce one or more secondary signals in a variety of intracellular signal transduction pathways, e.g., through phosphatidylinositol or cyclic AMP metabolism and turnover, in a cell. G proteins represent a family of heterotrimeric proteins composed of α, β and γ subunits, which bind guanine nucleotides. These proteins are usually linked to cell surface receptors, e.g., receptors containing seven transmembrane domains, such as the ligand receptors. Following ligand binding to the receptor, a conformational change is transmitted to the G protein, which causes the α-subunit to exchange a bound GDP molecule for a GTP molecule and to dissociate from the βγ-subunits. The GTP-bound form of the α-subunit typically functions as an effector-modulating moiety, leading to the production of second messengers, such as cyclic AMP (e.g., by activation of adenylate cyclase), diacylglycerol or inositol phosphates. Greater than 20 different types of α-subunits are known in man, which associate with a smaller pool of β and γ subunits. Examples of mammalian G proteins include Gi, Go, Gq, Gs and Gt. G proteins are described extensively in Lodish H. et al. Molecular Cell Biology, (Scientific American Books Inc., New York, N.Y., 1995), the contents of which are incorporated herein by reference.

As used herein, the phrase “phosphatidylinositol turnover and metabolism” includes the molecules involved in the turnover and metabolism of phosphatidylinositol 4,5-bisphosphate (PIP₂) as well as to the activities of these molecules. PIP₂ is a phospholipid found in the cytosolic leaflet of the plasma membrane. Binding of a ligand to the SLGP activates, in some cells, the plasma-membrane enzyme phospholipase C that in turn can hydrolyze PIP₂ to produce 1,2-diacylglycerol (DAG) and inositol 1,4,5-triphosphate (IP₃). Once formed IP₃ can diffuse to the endoplasmic reticulum surface where it can bind an IP₃ receptor, e.g., a calcium channel protein containing an IP₃ binding site. IP₃ binding can induce opening of the channel, allowing calcium ions to be released into the cytoplasm. IP₃ can also be phosphorylated by a specific kinase to form inositol 1,3,4,5-tetraphosphate (IP₄), a molecule which can cause calcium entry into the cytoplasm from the extracellular medium. IP₃ and IP4 can subsequently be hydrolyzed very rapidly to the inactive products inositol 1,4-biphosphate (IP₂) and inositol 1,3,4-triphosphate, respectively. These inactive products can be recycled by the cell to synthesize PIP₂. The other second messenger produced by the hydrolysis of PIP₂, namely 1,2-diacylglycerol (DAG), remains in the cell membrane where it can serve to activate the enzyme protein kinase C. Protein kinase C is usually found soluble in the cytoplasm of the cell, but upon an increase in the intracellular calcium concentration, this enzyme can move to the plasma membrane where it can be activated by DAG. The activation of protein kinase C in different cells results in various cellular responses such as the phosphorylation of glycogen synthase, or the phosphorylation of various transcription factors, e.g., NF-kB. The language “phosphatidylinositol activity”, as used herein, includes an activity of PIP₂ or one of its metabolites.

Another signaling pathway in which the SLGP protein may participate is the cAMP turnover pathway. As used herein, “cyclic AMP turnover and metabolism” includes molecules involved in the turnover and metabolism of cyclic AMP (cAMP) as well as to the activities of these molecules. Cyclic AMP is a second messenger produced in response to ligand induced stimulation of certain G protein coupled receptors. In the ligand signaling pathway, binding of ligand to a ligand receptor can lead to the activation of the enzyme adenylate cyclase, which catalyzes the synthesis of cAMP. The newly synthesized cAMP can in turn activate a cAMP-dependent protein kinase.

Preferred SLGP molecules of the present invention have an amino acid sequence sufficiently homologous to the amino acid sequence of SEQ ID NO:2. As used herein, the term “sufficiently homologous” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains and/or a common functional activity. For example, amino acid or nucleotide sequences which share common structural domains have at least about 50% homology, preferably 60% homology, more preferably 70%-80%, and even more preferably 90-95% homology across the amino acid sequences of the domains and contain at least one and preferably two structural domains, are defined herein as sufficiently homologous. Furthermore, amino acid or nucleotide sequences which share at least 50%, preferably 60%, more preferably 70-80, or 90-95% homology and share a common functional activity are defined herein as sufficiently homologous.

As used interchangeably herein, an “SLGP activity”, “biological activity of SLGP” or “functional activity of SLGP”, refers to an activity exerted by a SLGP protein, polypeptide or nucleic acid molecule on a SLGP responsive cell as determined in vivo, or in vitro, according to standard techniques. In one embodiment, a SLGP activity is a direct activity, such as an association with a SLGP-traget molecule. As used herein, a “target molecule” or “binding partner” is a molecule with which a SLGP protein binds or interacts in nature, such that SLGP-mediated function is acheived. An SLGP target molecule can be a non-SLGP molecule or a SLGP protein or polypeptide of the present invention. In an exemplary embodiment, a SLGP target molecule is a SLGP ligand. Alternatively, a SLGP activity is an indirect activity, such as a cellular signaling activity mediated by interaction of the SLGP protein with a SLGP ligand.

In a preferred embodiment, a SLGP activity is at least one or more of the following activities: (i) interaction of a SLGP protein with soluble SLGP ligand (e.g., CD55); (ii) interaction of a SLGP protein with a membrane-bound non-SLGP protein; (iii) interaction of a SLGP protein with an intracellular protein (e.g., an intracellular enzyme or signal transduction molecule); and (iv) indirect interaction of a SLGP protein with an intracellular protein (e.g., a downstream signal transduction molecule).

In yet another preferred embodiment, a SLGP activity is at least one or more of the following activities: (1) modulation of cellular signal transduction, either in vitro or in vivo; (2) regulation of activation in a cell expressing a SLGP protein (e.g., leukocyte activation); (3) regulation of a hematopoietic cell expressing a SLGP protein, wherein said hematopoietic cell is involved in inflammation; (4) regulation of small synaptic vesicle exocytosis (e.g., small synaptic vesicle exocytosis in neurons in response to exposure to alpha-latrotoxin); (5) regulation of inflammation.

Accordingly, another embodiment of the invention features isolated SLGP proteins and polypeptides having a SLGP activity. Preferred SLGP proteins have at least one transmembrane domain and a SLGP activity. In a preferred embodiment, a SLGP protein has a 7 transmembrane receptor profile and a SLGP activity. In another preferred embodiment, a SLGP protein has a EGF-like domain and a SLGP activity. In another preferred embodiment, a SLGP protein has an NADH-ubiquinone/plastoquinone oxidoreductase chain 4L domain and a SLGP activity. In another preferred embodiment, a SLGP protein has a signal sequence and a SLGP activity. In still another preferred embodiment, a SLGP protein has a 7 transmembrane receptor profile, a EGF-like domain, and SLGP activity. In still another preferred embodiment, a SLGP protein has a 7 transmembrane receptor profile, a EGF-like domain, and an NADH-ubiquinone/plastoquinone oxidoreductase chain 4L domain and a SLGP activity. In still another preferred embodiment, a SLGP protein has a 7 transmembrane receptor profile and an NADH-ubiquinone/plastoquinone oxidoreductase chain 4L domain and a SLGP activity. In still another preferred embodiment, a SLGP protein has a EGF-like domain and an NADH-ubiquinone/plastoquinone oxidoreductase chain 4L domain and a SLGP activity. In still another preferred embodiment, a SLGP protein has a 7 transmembrane receptor profile, a EGF-like domain, a SLGP activity, and an amino acid sequence sufficiently homologous to an amino acid sequence of SEQ ID NO:2.

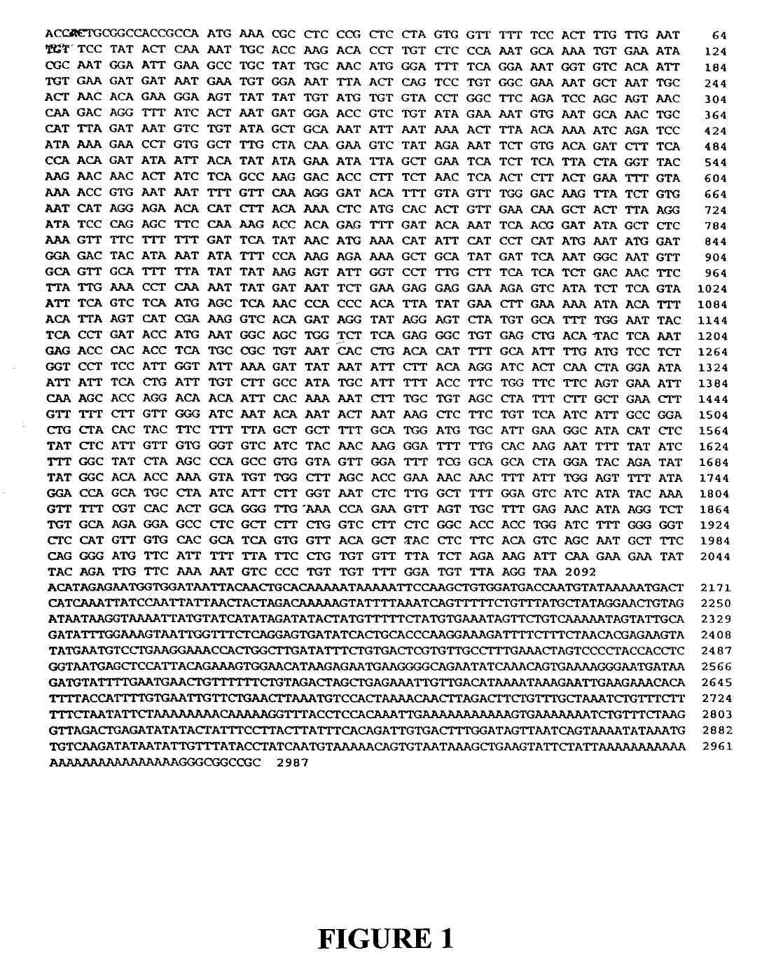

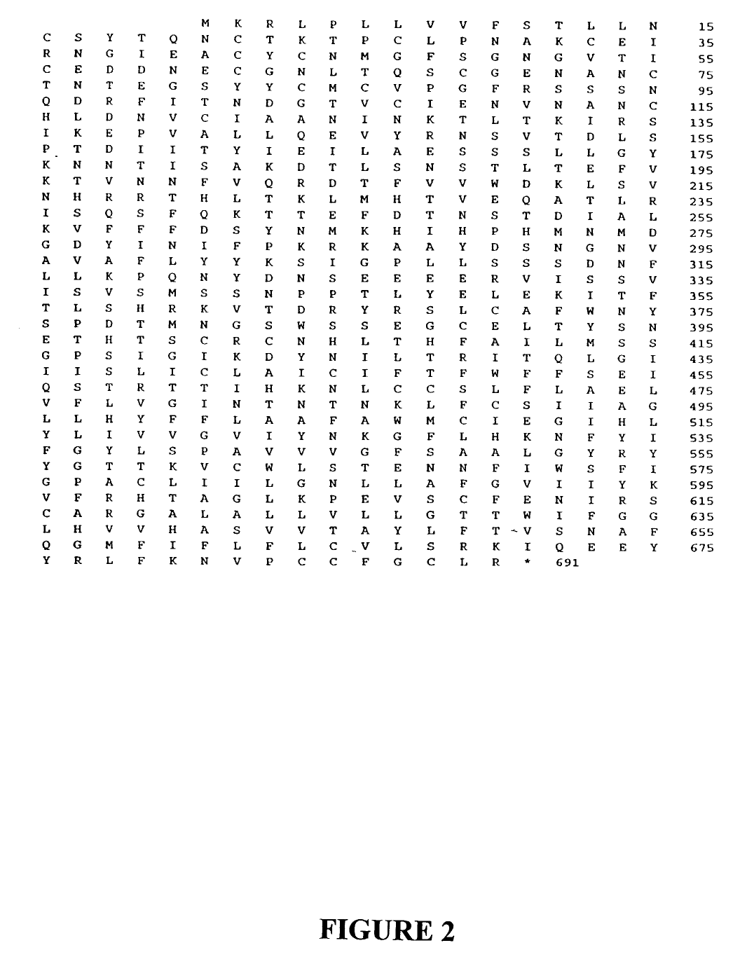

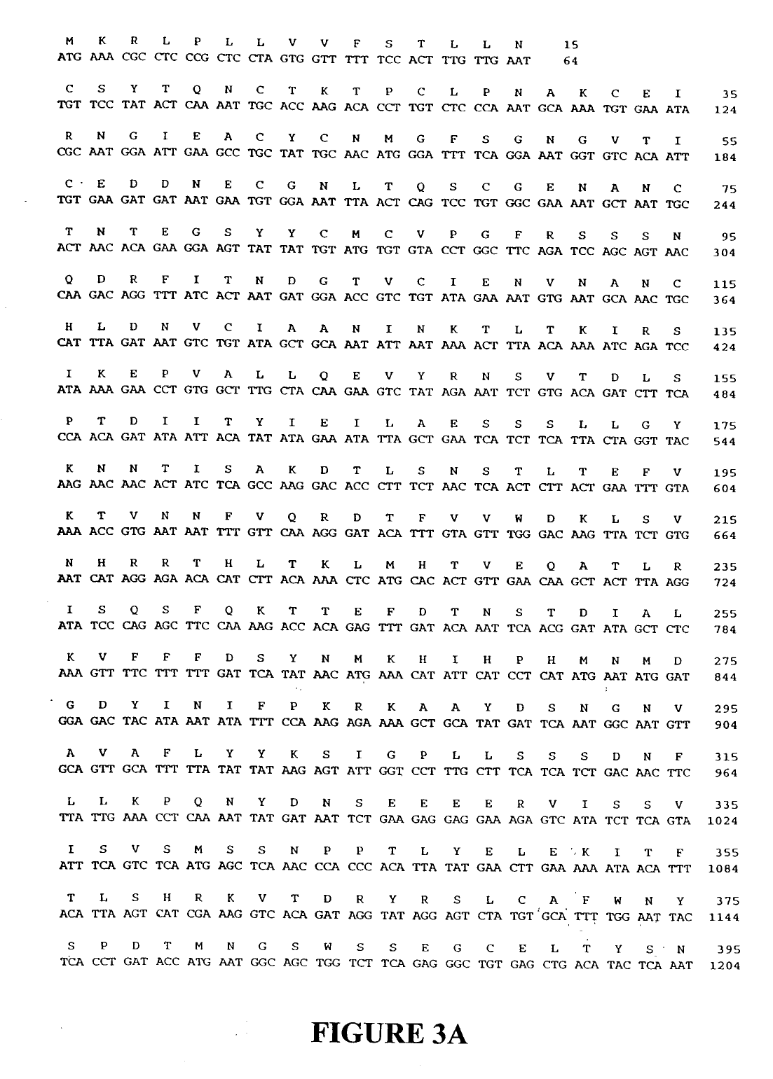

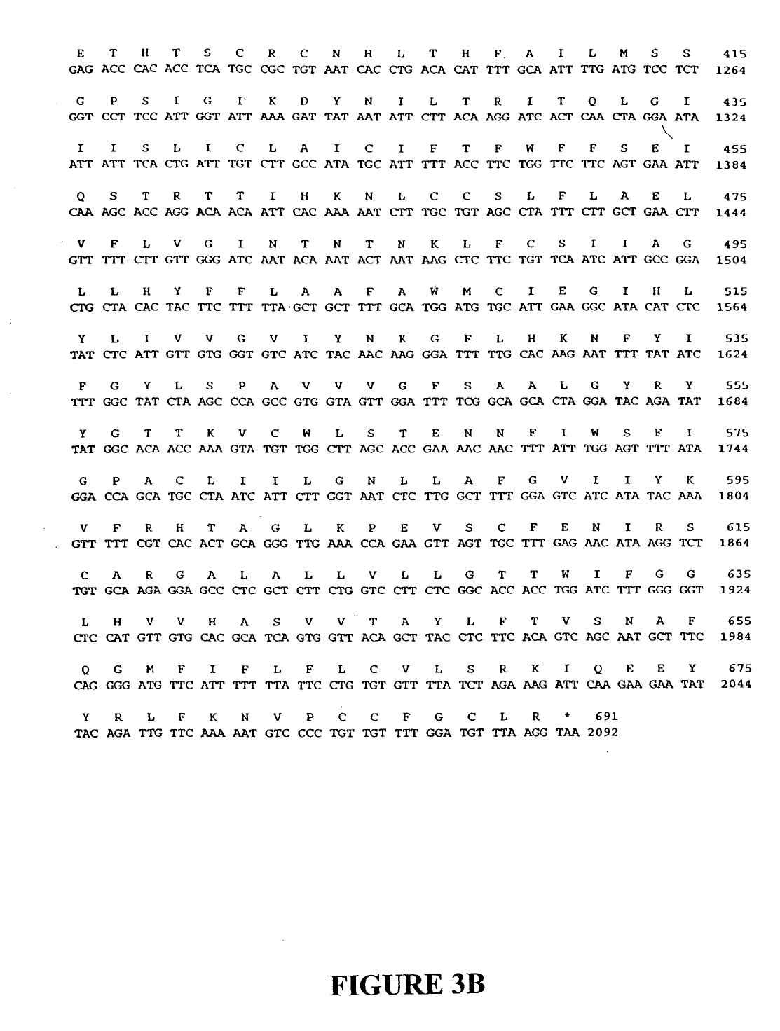

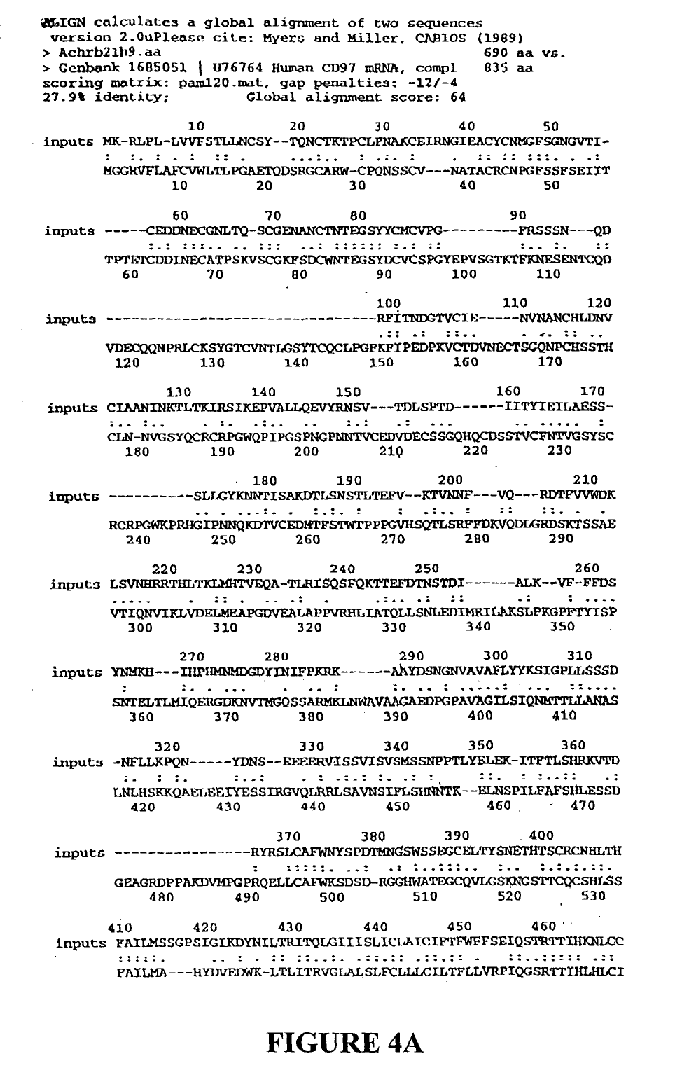

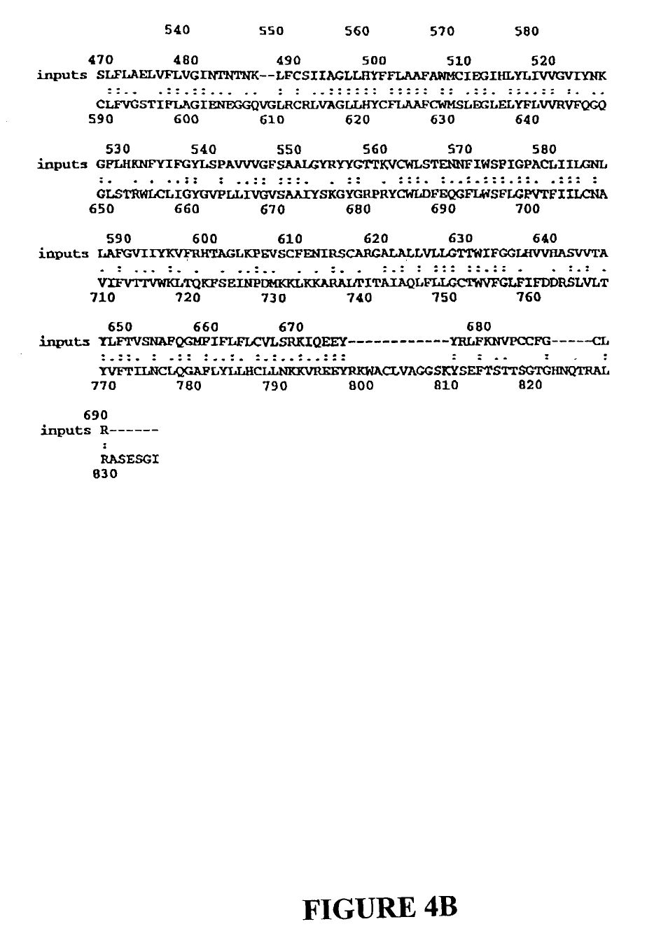

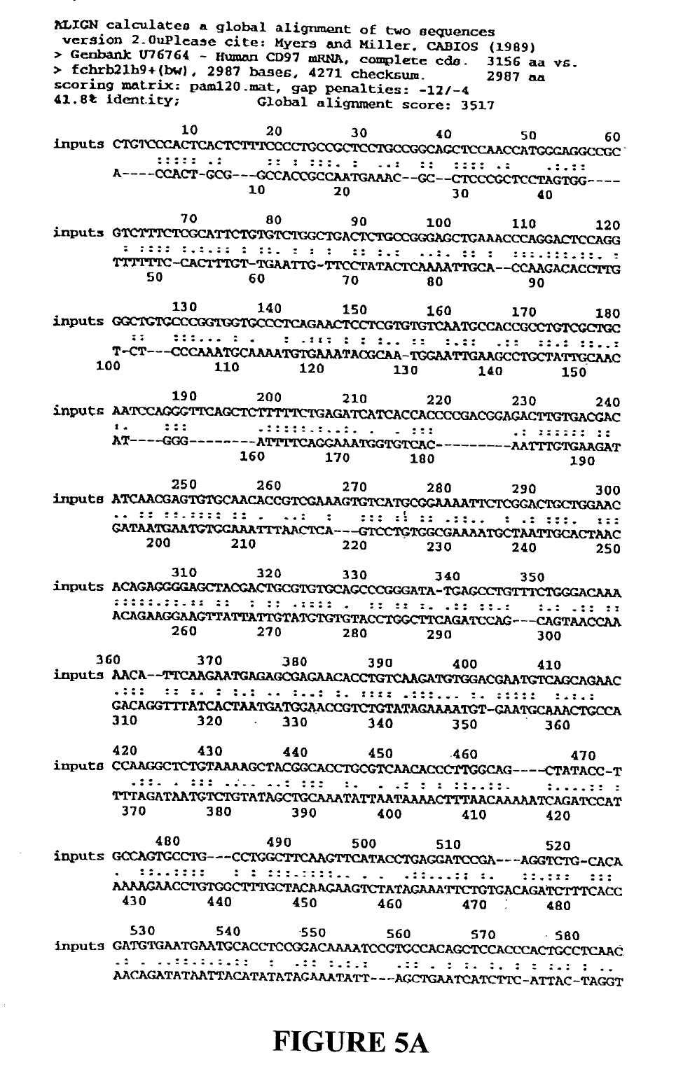

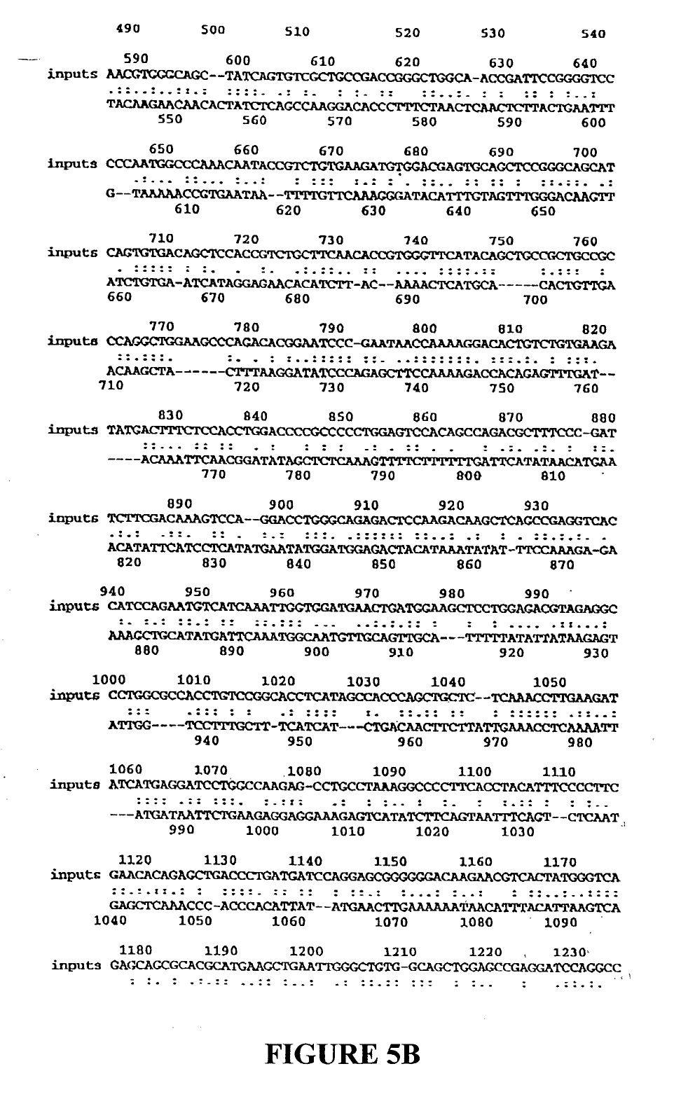

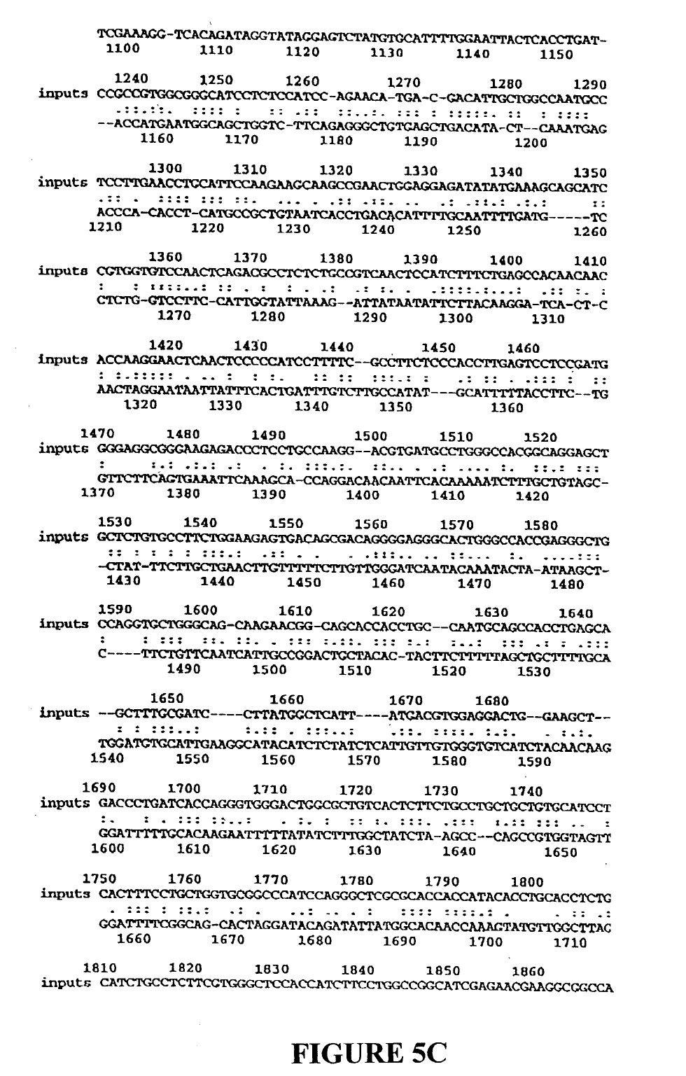

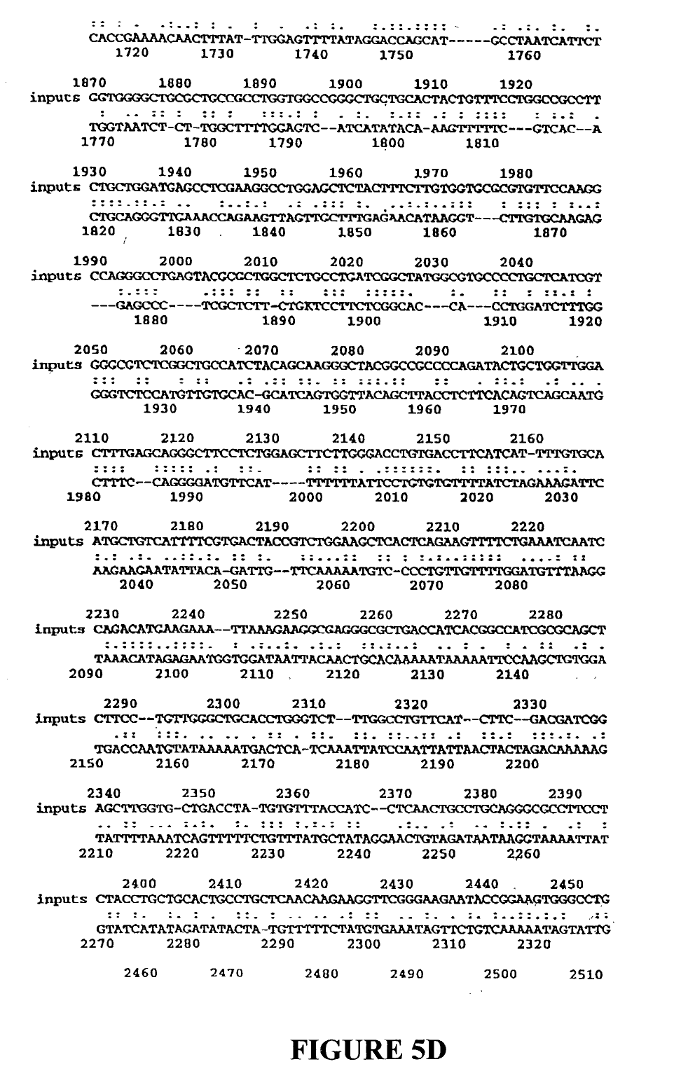

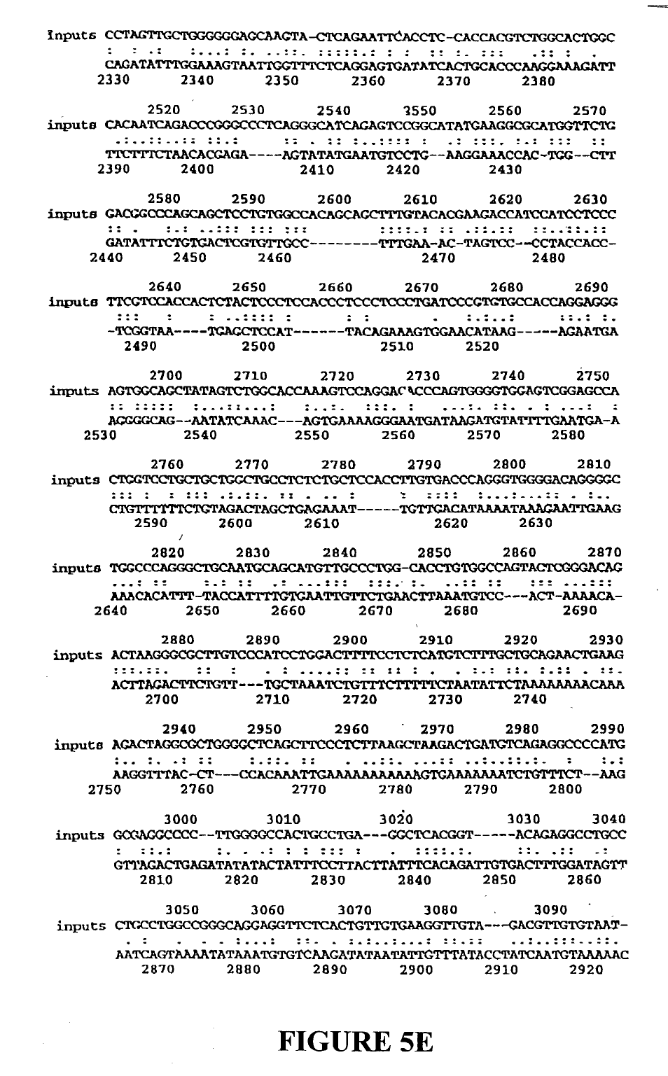

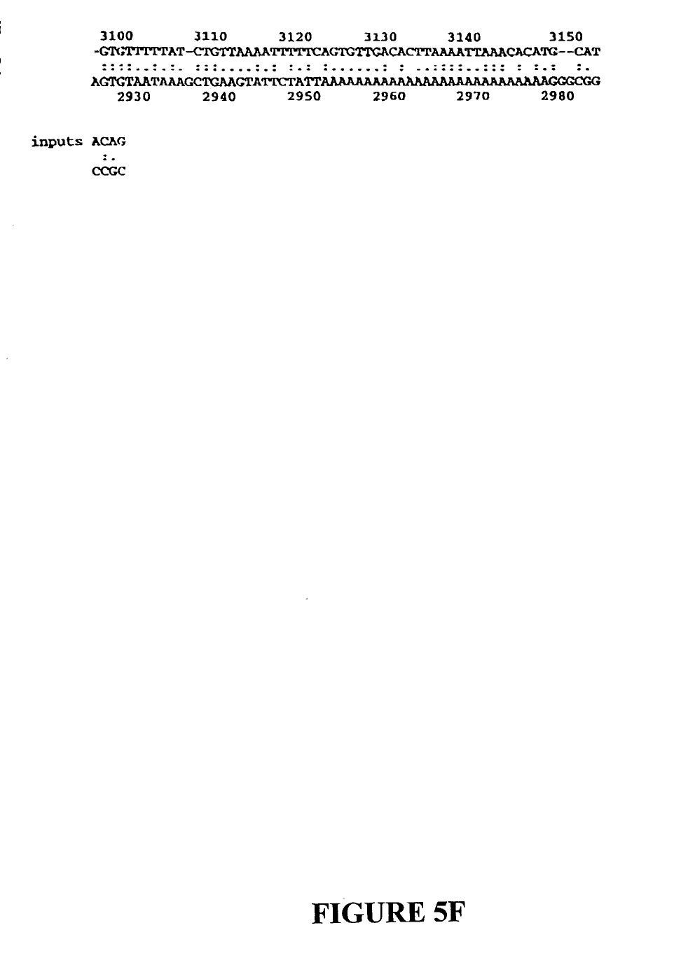

The nucleotide sequence of the isolated human SLGP cDNA and the predicted amino acid sequence of the human SLGP polypeptide are shown in FIG. 1 and in SEQ ID NOs: 1 and 2, respectively. A plasmid containing the nucleotide sequence encoding human SLGP was deposited with the American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

The human SLGP cDNA, which is approximately 2987 nucleotides in length, encodes a protein which is approximately 690 amino acid residues in length. The human SLGP protein contains 7 transmembrane domains at about amino acids 433-452, 465-481, 500-524, 533-553, 570-594, 619-635, and 642-666 of SEQ ID NO:2. The human SLGP protein further contains a 7 transmembrane receptor profile. The 7 transmembrane receptor profile can be found at least, for example, from about amino acids 421-678 of SEQ ID NO:2.

Various aspects of the invention are described in further detail in the following subsections:

I. Isolated Nucleic Acid Molecules

One aspect of the invention pertains to isolated nucleic acid molecules that encode SLGP proteins or biologically active portions thereof, as well as nucleic acid fragments sufficient for use as hybridization probes to identify SLGP-encoding nucleic acids (e.g., SLGP mRNA) and fragments for use as PCR primers for the amplification or mutation of SLGP nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from chromosomal DNA, e.g., other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated SLGP nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1, the nucleotide sequence of the DNA insert of the plasmid deposited with ATCC as Accession Number ______, the nucleotide sequence of the DNA insert of the plasmid deposited with ATCC as Accession Number ______, or a portion thereof, can be isolated using standard molecular biology techniques and the sequence information provided herein. Using all or portion of the nucleic acid sequence of SEQ ID NO:1, the nucleotide sequence of the DNA insert of the plasmid deposited with ATCC as Accession Number ______, or the nucleotide sequence of the DNA insert of the plasmid deposited with ATCC as Accession Number ______ as a hybridization probe, SLGP nucleic acid molecules can be isolated using standard hybridization and cloning techniques (e.g., as described in Sambrook, J., Fritsh, E. F., and Maniatis, T. Molecular Cloning: A Laboratory Manual. 2nd, ed, Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, the nucleotide sequence of the DNA insert of the plasmid deposited with ATCC as Accession Number ______, or the nucleotide sequence of the DNA insert of the plasmid deposited with ATCC as Accession Number ______ can be isolated by the polymerase chain reaction (PCR) using synthetic oligonucleotide primers designed based upon the sequence of SEQ ID NO:1, the nucleotide sequence of the DNA insert of the plasmid deposited with ATCC as Accession Number ______, or the nucleotide sequence of the DNA insert of the plasmid deposited with ATCC as Accession Number ______.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to SLGP nucleotide sequences can be prepared by standard synthetic techniques, e.g., using an automated DNA synthesizer.

In a preferred embodiment, an isolated nucleic acid molecule of the invention comprises the nucleotide sequence shown in SEQ ID NO:1. The sequence of SEQ ID NO:1 corresponds to the human SLGP cDNA. This cDNA comprises sequences encoding the human SLGP protein (i.e., “the coding region”, from nucleotides 1-2070), as well as 3′ untranslated sequences (nucleotides 2071-2987). Alternatively, the nucleic acid molecule can comprise only the coding region of SEQ ID NO:1 (e.g., nucleotides 1-2070, corresponding to SEQ ID NO:3).

In another preferred embodiment, an isolated nucleic acid molecule of the invention comprises a nucleic acid molecule which is a complement of the nucleotide sequence shown in SEQ ID NO:1, SEQ ID NO:3, or the nucleotide sequence of the DNA insert of the plasmid deposited with ATCC as Accession Number ______, or a portion of any of these nucleotide sequences. A nucleic acid molecule which is complementary to the nucleotide sequence shown in SEQ ID NO:1, SEQ ID NO:3, or the nucleotide sequence of the DNA insert of the plasmid deposited with ATCC as Accession Number ______, is one which is sufficiently complementary to the nucleotide sequence shown in SEQ ID NO:1, SEQ ID NO:3, or the nucleotide sequence of the DNA insert of the plasmid deposited with ATCC as Accession Number ______, such that it can hybridize to the nucleotide sequence shown in SEQ ID NO:1, SEQ ID NO:3, or the nucleotide sequence of the DNA insert of the plasmid deposited with ATCC as Accession Number ______, thereby forming a stable duplex.

In still another preferred embodiment, an isolated nucleic acid molecule of the present invention comprises a nucleotide sequence which is at least about 40%, 42%, 45%, 50%, 55%, 60%, 65%, 70%, 75%, 80%, 85%, 90%, 95%, 98% or more homologous to the entire length of the nucleotide sequence shown in SEQ ID NO:1, SEQ ID NO:3, or the entire length of the nucleotide sequence of the DNA insert of the plasmid deposited with ATCC as Accession Number ______, or a portion of any of these nucleotide sequences.

Moreover, the nucleic acid molecule of the invention can comprise only a portion of the nucleic acid sequence of SEQ ID NO:1, SEQ ID NO:3, or the nucleotide sequence of the DNA insert of the plasmid deposited with ATCC as Accession Number ______, for example, a fragment which can be used as a probe or primer or a fragment encoding a biologically active portion of an SLGP protein. The nucleotide sequence determined from the cloning of the SLGP gene allows for the generation of probes and primers designed for use in identifying and/or cloning other SLGP family members, as well as SLGP homologues from other species. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1, SEQ ID NO:3, or the nucleotide sequence of the DNA insert of the plasmid deposited with ATCC as Accession Number ______, of an anti-sense sequence of SEQ ID NO:1, SEQ ID NO:3, or the nucleotide sequence of the DNA insert of the plasmid deposited with ATCC as Accession Number ______, or of a naturally occurring allelic variant or mutant of SEQ ID NO:1, SEQ ID NO:3, or the nucleotide sequence of the DNA insert of the plasmid deposited with ATCC as Accession Number ______. In an exemplary embodiment, a nucleic acid molecule of the present invention comprises a nucleotide sequence which is greater than 488, or more nucleotides in length and hybridizes under stringent hybridization conditions to a nucleic acid molecule of SEQ ID NO:1, SEQ ID NO:3, or the nucleotide sequence of the DNA insert of the plasmid deposited with ATCC as Accession Number ______.

Probes based on the SLGP nucleotide sequences can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a SLGP protein, such as by measuring a level of a SLGP-encoding nucleic acid in a sample of cells from a subject e.g., detecting SLGP mRNA levels or determining whether a genomic SLGP gene has been mutated or deleted.

A nucleic acid fragment encoding a “biologically active portion of a SLGP protein” can be prepared by isolating a portion of the nucleotide sequence of SEQ ID NO:1, the nucleotide sequence of the DNA insert of the plasmid deposited with ATCC as Accession Number ______, or the nucleotide sequence of the DNA insert of the plasmid deposited with ATCC as Accession Number ______, which encodes a polypeptide having a SLGP biological activity (the biological activities of the SLGP proteins have previously been described), expressing the encoded portion of the SLGP protein (e.g., by recombinant expression in vitro) and assessing the activity of the encoded portion of the SLGP protein.

The invention further encompasses nucleic acid molecules that differ from the nucleotide sequence shown in SEQ ID NO:1, SEQ ID NO:3, or the nucleotide sequence of the DNA insert of the plasmid deposited with ATCC as Accession Number ______, due to degeneracy of the genetic code and thus encode the same SLGP proteins as those encoded by the nucleotide sequence shown in SEQ ID NO:1, SEQ ID NO:3, or the nucleotide sequence of the DNA insert of the plasmid deposited with ATCC as Accession Number ______. In another embodiment, an isolated nucleic acid molecule of the invention has a nucleotide sequence encoding a protein having an amino acid sequence shown in SEQ ID NO:2.

In addition to the SLGP nucleotide sequences shown in SEQ ID NO:1, SEQ ID NO:3, or the nucleotide sequence of the DNA insert of the plasmid deposited with ATCC as Accession Number ______, it will be appreciated by those skilled in the art that DNA sequence polymorphisms that lead to changes in the amino acid sequences of the SLGP proteins may exist within a population (e.g., the human population). Such genetic polymorphism in the SLGP genes may exist among individuals within a population due to natural allelic variation. As used herein, the terms “gene” and “recombinant gene” refer to nucleic acid molecules isolated from chromosomal DNA, which include an open reading frame encoding an SLGP protein, preferably a mammalian SLGP protein. A gene includes coding DNA sequences, non-coding regulatory sequences, and introns. As used herein, a gene refers to an isolated nucleic acid molecule, as defined herein.

Allelic variants of human SLGP include both functional and non-functional SLGP proteins. Functional allelic variants are naturally occurring amino acid sequence variants of the humanSLGP protein that maintain the ability to bind an SLGP ligand and/or modulate programmed cell death. Functional allelic variants will typically contain only conservative substitution of one or more amino acids of SEQ ID NO:2 or substitution, deletion or insertion of non-critical residues in non-critical regions of the protein.

Non-functional allelic variants are naturally occurring amino acid sequence variants of the human SLGP protein that do not have the ability to either bind an SLGP ligand and/or modulate programmed cell death. Non-functional allelic variants will typically contain a non-conservative substitution, a deletion, or insertion or premature truncation of the amino acid sequence of SEQ ID NO:2 or a substitution, insertion or deletion in critical residues or critical regions.

The present invention further provides non-human orthologues of the human SLGP protein. Orthologues of the human SLGP protein are proteins that are isolated from non-human organisms and possess the same SLGP ligand binding and/or modulation of programmed cell death capabilities of the human SLGP protein. Orthologues of the human SLGP protein can readily be identified as comprising an amino acid sequence that is substantially homologous to SEQ ID NO:2.

Moreover, nucleic acid molecules encoding other GPCR family members (e.g., other SLGP family members) and thus which have a nucleotide sequence which differs from the SLGP sequences of SEQ ID NO:1, SEQ ID NO:3, or the nucleotide sequence of the DNA insert of the plasmid deposited with ATCC as Accession Number ______ are intended to be within the scope of the invention. For example, another SLGP cDNA can be identified based on the nucleotide sequence of human SLGP. Moreover, nucleic acid molecules encoding SLOP proteins from different species, and which, thus, have a nucleotide sequence which differs from the SLGP sequences of SEQ ID NO:1, SEQ ID NO:3, or the nucleotide sequence of the DNA insert of the plasmid deposited with ATCC as Accession Number ______ are intended to be within the scope of the invention. For example, a mouse SLGP cDNA can be identified based on the nucleotide sequence of a human SLGP.

Nucleic acid molecules corresponding to natural allelic variants and homologues of the SLGP cDNAs of the invention can be isolated based on their homology to the SLGP nucleic acids disclosed herein using the cDNAs disclosed herein, or a portion thereof, as a hybridization probe according to standard hybridization techniques under stringent hybridization conditions.

Accordingly, in another embodiment, an isolated nucleic acid molecule of the invention is at least 15 nucleotides in length and hybridizes under stringent conditions to the nucleic acid molecule comprising the nucleotide sequence of SEQ ID NO:1, SEQ ID NO:3, the nucleotide sequence of the DNA insert of the plasmid deposited with ATCC as Accession Number ______, or the nucleotide sequence of the DNA insert of the plasmid deposited with ATCC as Accession Number ______. In another embodiment, the nucleic acid is at least 30, 50, 100, 150, 200, 250, 275, 300, 350, 400, 450, 500, 550, 600, 650, 700, 750, 800, 850, 900, or 950 nucleotides in length. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 70%, more preferably at least about 80%, even more preferably at least about 85% or 90% homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2× SSC, 0.1% SDS at 50-65° C. Preferably, an isolated nucleic acid molecule of the invention that hybridizes under stringent conditions to the sequence of SEQ ID NO:1, the nucleotide sequence of the DNA insert of the plasmid deposited with ATCC as Accession Number ______, or the nucleotide sequence of the DNA insert of the plasmid deposited with ATCC as Accession Number ______, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the SLGP sequences that may exist in the population, the skilled artisan will further appreciate that changes can be introduced by mutation into the nucleotide sequences of SEQ ID NO:1, SEQ ID NO:3, or the nucleotide sequence of the DNA insert of the plasmid deposited with ATCC as Accession Number ______, thereby leading to changes in the amino acid sequence of the encoded SLGP proteins, without altering the functional ability of the SLGP proteins. For example, nucleotide substitutions leading to amino acid substitutions at “non-essential” amino acid residues can be made in the sequence of SEQ ID NO:1, SEQ ID NO:3, or the nucleotide sequence of the DNA insert of the plasmid deposited with ATCC as Accession Number ______. A “non-essential” amino acid residue is a residue that can be altered from the wild-type sequence of SLGP (e.g., the sequence of SEQ ID NO:2) without altering the biological activity, whereas an “essential” amino acid residue is required for biological activity. For example, amino acid residues that are conserved among the SLGP proteins of the present invention, are predicted to be particularly unamenable to alteration. Furthermore, additional amino acid residues that are conserved between the SLGP proteins of the present invention and other members of the GPCR families are not likely to be amenable to alteration.

Accordingly, another aspect of the invention pertains to nucleic acid molecules encoding SLGP proteins that contain changes in amino acid residues that are not essential for activity. Such SLGP proteins differ in amino acid sequence from SEQ ID NO:2, yet retain biological activity. In one embodiment, the isolated nucleic acid molecule comprises a nucleotide sequence encoding a protein, wherein the protein comprises an amino acid sequence at least about 25%, 28%, 30%, 35%, 40%, 45%, 50%, 55%, 60%, 65%, 70%, 75%, 80%, 85%, 90%, 95%, 98% or more homologous to SEQ ID NO:2.

An isolated nucleic acid molecule encoding an SLGP protein homologous to the protein of SEQ ID NO:2 can be created by introducing one or more nucleotide substitutions, additions or deletions into the nucleotide sequence of SEQ ID NO:1, SEQ ID NO:3, or the nucleotide sequence of the DNA insert of the plasmid deposited with ATCC as Accession Number ______, such that one or more amino acid substitutions, additions or deletions are introduced into the encoded protein. Mutations can be introduced into SEQ ID NO:1, SEQ ID NO:3, or the nucleotide sequence of the DNA insert of the plasmid deposited with ATCC as Accession Number ______ by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an SLGP protein is preferably replaced with another amino acid residue from the same side chain family. Alternatively, in another embodiment, mutations can be introduced randomly along all or part of an SLGP coding sequence, such as by saturation mutagenesis, and the resultant mutants can be screened for SLGP biological activity to identify mutants that retain activity. Following mutagenesis of SEQ ID NO:1, SEQ ID NO:3, or the nucleotide sequence of the DNA insert of the plasmid deposited with ATCC as Accession Number ______, the encoded protein can be expressed recombinantly and the activity of the protein can be determined.

In a preferred embodiment, a mutant SLGP protein can be assayed for the ability to affect the (1) modulation of cellular signal transduction, either in vitro or in vivo; (2) regulation of activation in a cell expressing a SLGP protein (e.g., leukocyte activation); (3) regulation of a hematopoietic cell expressing a SLGP protein, wherein said hematopoietic cell is involved in inflammation; (4) regulation of small synaptic vesicle exocytosis (e.g., small synaptic vesicle exocytosis in neurons in response to exposure to alpha-latrotoxin); (5) regulation of inflammation.

In addition to the nucleic acid molecules encoding SLGP proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire SLGP coding strand, or to only a portion thereof. In one embodiment, an antisense nucleic acid molecule is antisense to a “coding region” of the coding strand of a nucleotide sequence encoding SLGP. The term “coding region” refers to the region of the nucleotide sequence comprising codons which are translated into amino acid residues (e.g., the coding region of human SLGP corresponds to SEQ ID NO:3). In another embodiment, the antisense nucleic acid molecule is antisense to a “noncoding region” of the coding strand of a nucleotide sequence encoding SLGP. The term “noncoding region” refers to 5′ and 3′ sequences which flank the coding region that are not translated into amino acids (i.e., also referred to as 5′ and 3′ untranslated regions).

Given the coding strand sequences encoding SLGP disclosed herein (e.g., SEQ ID NO:3), antisense nucleic acids of the invention can be designed according to the rules of Watson and Crick base pairing. The antisense nucleic acid molecule can be complementary to the entire coding region of SLGP mRNA, but more preferably is an oligonucleotide which is antisense to only a portion of the coding or noncoding region of SLGP mRNA. For example, the antisense oligonucleotide can be complementary to the region surrounding the translation start site of SLGP mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine,

2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)_{w}, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a SLGP protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave SLGP mRNA transcripts to thereby inhibit translation of SLGP mRNA. A ribozyme having specificity for a SLGP-encoding nucleic acid can be designed based upon the nucleotide sequence of a SLGP cDNA disclosed herein (i.e., SEQ ID NO:1). For example, a derivative of a Tetrahymena L-19 IVS RNA can be constructed in which the nucleotide sequence of the active site is complementary to the nucleotide sequence to be cleaved in a SLGP-encoding mRNA. See, e.g., Cech et al. U.S. Pat. No. 4,987,071; and Cech et al. U.S. Pat. No. 5,116,742. Alternatively, SLGP mRNA can be used to select a catalytic RNA having a specific ribonuclease activity from a pool of RNA molecules. See, e.g., Bartel, D. and Szostak, J. W. (1993) Science 261:1411-1418.

Alternatively, SLGP gene expression can be inhibited by targeting nucleotide sequences complementary to the regulatory region of the SLGP (e.g., the SLGP promoter and/or enhancers) to form triple helical structures that prevent transcription of the SLGP gene in target cells. See generally, Helene, C. (1991) Anticancer Drug Des. 6(6):569-84; Helene, C. et al. (1992) Ann. N. Y Acad. Sci. 660:27-36; and Maher, L. J. (1992) Bioassays 14(12):807-15.

In yet another embodiment, the SLGP nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. PNAS 93: 14670-675.

PNAs of SLGP nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of SLGP nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of SLGP can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of SLGP nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. US. 86:6553-6556; Lemaitre et al. (1987) Proc. Natl. Acad. Sci. USA 84:648-652; PCT Publication No. WO88/09810, published Dec. 15, 1988) or the blood-brain barrier (see, e.g., PCT Publication No. WO89/10134, published Apr. 25, 1988). In addition, oligonucleotides can be modified with hybridization-triggered cleavage agents (See, e.g., Krol et al. (1988) BioTechniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Furthermore, given the fact that an important use for the SLGP molecules of the present invention is in the screening for SLGP ligands (e.g., surrogate ligands) and/or SLGP modulators, it is intended that the following are also within the scope of the present invention: isolated nucleic acids which encode and SLGP ligands or SLGP modulators, probes and/or primers useful for identifying SLGP ligands or SLGP modulators based on the sequences of nucleic acids which encode and SLGP ligands or SLGP modulators, isolated nucleic acid molecules which are complementary or antisense to the sequences of nucleic acids which encode and SLGP ligands or SLGP modulators, isolated nucleic acid molecules which are at least about 60-65%, preferably at least about 70-75%, more preferable at least about 80-85%, and even more preferably at least about 90-95% or more homologous to the sequences of nucleic acids which encode and SLGP ligands or SLGP modulators, portions of nucleic acids which encode and SLGP ligands or SLGP modulators (e.g., biologically-active portions), naturally-occurring allelic variants of nucleic acids which encode and SLGP ligands or SLGP modulators, nucleic acid molecules which hybridize under stringent hybridization conditions to nucleic acids which encode and SLGP ligands or SLGP modulators, functionally-active mutants of nucleic acids which encode and SLGP ligands or SLGP modulators, PNAs of nucleic acids which encode and SLGP ligands or SLGP modulators, as well as vectors containing a nucleic acid encoding a SLGP ligand or SLGP modulator, described herein, host cells into which an expression vector encoding a SLGP ligand or SLGP modulator has been introduced, and homologous recombinant animal which express SLGP ligands or SLGP modulators.

II. Isolated SLGP Proteins and Anti-SLGP Antibodies

One aspect of the invention pertains to isolated SLGP proteins, and biologically active portions thereof, as well as polypeptide fragments suitable for use as immunogens to raise anti-SLGP antibodies. In one embodiment, native SLGP proteins can be isolated from cells or tissue sources by an appropriate purification scheme using standard protein purification techniques. In another embodiment, SLGP proteins are produced by recombinant DNA techniques. Alternative to recombinant expression, a SLGP protein or polypeptide can be synthesized chemically using standard peptide synthesis techniques.

An “isolated” or “purified” protein or biologically active portion thereof is substantially free of cellular material or other contaminating proteins from the cell or tissue source from which the SLGP protein is derived, or substantially free from chemical precursors or other chemicals when chemically synthesized. The language “substantially free of cellular material” includes preparations of SLGP protein in which the protein is separated from cellular components of the cells from which it is isolated or recombinantly produced. In one embodiment, the language “substantially free of cellular material” includes preparations of SLGP protein having less than about 30% (by dry weight) of non-SLGP protein (also referred to herein as a “contaminating protein”), more preferably less than about 20% of non-SLGP protein, still more preferably less than about 10% of non-SLGP protein, and most preferably less than about 5% non-SLGP protein. When the SLGP protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of SLGP protein in which the protein is separated from chemical precursors or other chemicals which are involved in the synthesis of the protein. In one embodiment, the language “substantially free of chemical precursors or other chemicals” includes preparations of SLGP protein having less than about 30% (by dry weight) of chemical precursors or non-SLGP chemicals, more preferably less than about 20% chemical precursors or non-SLGP chemicals, still more preferably less than about 10% chemical precursors or non-SLGP chemicals, and most preferably less than about 5% chemical precursors or non-SLGP chemicals.

Biologically active portions of a SLGP protein include peptides comprising amino acid sequences sufficiently homologous to or derived from the amino acid sequence of the SLGP protein, e.g., the amino acid sequence shown in SEQ ID NO:2, which include less amino acids than the full length SLGP proteins, and exhibit at least one activity of a SLGP protein. Typically, biologically active portions comprise a domain or motif with at least one activity of the SLGP protein. A biologically active portion of a SLGP protein can be a polypeptide which is, for example, 10, 25, 50, 100 or more amino acids in length.

In one embodiment, a biologically active portion of a SLGP protein comprises at least a transmembrane domain. In another embodiment, a biologically active portion of a SLGP protein comprises at least one 7 transmembrane receptor profile. In another embodiment, a biologically active portion of a SLGP protein comprises at least an EGF-like domain. In another embodiment, a biologically active portion of a SLGP protein comprises at least an NADH-ubiquinone/plastoquinone oxidoreductase chain 4L domain. In another embodiment, a biologically active portion of a SLGP protein comprises at least a signal sequence. In another embodiment a biologically active portion of a SLGP protein comprises at least a 7 transmembrane receptor profile and an EGF-like domain. In another embodiment a biologically active portion of a SLGP protein comprises at least a 7 transmembrane receptor profile and an NADH-ubiquinone/plastoquinone oxidoreductase chain 4L domain. In another embodiment a biologically active portion of a SLGP protein comprises at least a 7 transmembrane receptor profile and a signal sequence. In another embodiment a biologically active portion of a SLGP protein comprises at least a an EGF-like domain and an NADH-ubiquinone/plastoquinone oxidoreductase chain 4L domain. In another embodiment a biologically active portion of a SLGP protein comprises at least a an EGF-like domain and a signal sequence. In another embodiment a biologically active portion of a SLGP protein comprises at least an NADH-ubiquinone/plastoquinone oxidoreductase chain 4L domain and a signal sequence. In another embodiment a biologically active portion of a SLGP protein comprises at least a 7 transmembrane receptor profile, an EGF-like domain and an NADH-ubiquinone/plastoquinone oxidoreductase chain 4L domain. In another embodiment a biologically active portion of a SLGP protein comprises at least a 7 transmembrane receptor profile, an EGF-like domain, an NADH-ubiquinone/plastoquinone oxidoreductase chain 4L domain, and a signal sequence.

It is to be understood that a preferred biologically active portion of a SLGP protein of the present invention may contain at least one of the above-identified structural domains and/or profiles. A more preferred biologically active portion of a SLGP protein may contain at least two of the above-identified structural domains and/or profiles. Moreover, other biologically active portions, in which other regions of the protein are deleted, can be prepared by recombinant techniques and evaluated for one or more of the functional activities of a native SLGP protein.

In a preferred embodiment, the SLGP protein has an amino acid sequence shown in SEQ ID NO:2. In other embodiments, the SLGP protein is substantially homologous to SEQ ID NO:2, and retains the functional activity of the protein of SEQ ID NO:2, yet differs in amino acid sequence due to natural allelic variation or mutagenesis, as described in detail in subsection I above. Accordingly, in another embodiment, the SLGP protein is a protein which comprises an amino acid sequence at least about 28%, 30%, 35%, 40%, 45%, 50%, 55%, 60%, 65%, 70%, 75%, 80%, 85%, 90%, 95%, 98% or more homologous to SEQ ID NO:2.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the SLGP amino acid sequence of SEQ ID NO:2 having 689 amino acid residues, at least 100, 200, preferably at least 300, more preferably at least 400, even more preferably at least 500, and even more preferably at least 600, 650 or 689 amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0) (available at http://DEBRA TO PROVIDE), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to SLGP nucleic acid molecules of the invention. BLAST protein searches can be performed with the XBLAST program, score=50, wordlength=3 to obtain amino acid sequences homologous to SLGP protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The invention also provides SLGP chimeric or fusion proteins. As used herein, a SLGP “chimeric protein” or “fusion protein” comprises a SLGP polypeptide operatively linked to a non-SLGP polypeptide. A “SLGP polypeptide” refers to a polypeptide having an amino acid sequence corresponding to SLGP, whereas a “non-SLGP polypeptide” refers to a polypeptide having an amino acid sequence corresponding to a protein which is not substantially homologous to the SLGP protein, e.g., a protein which is different from the SLGP protein and which is derived from the same or a different organism. Within a SLGP fusion protein the SLGP polypeptide can correspond to all or a portion of a SLGP protein. In a preferred embodiment, a SLGP fusion protein comprises at least one biologically active portion of a SLGP protein. In another preferred embodiment, a SLOP fusion protein comprises at least two biologically active portions of a SLGP protein. Within the fusion protein, the term “operatively linked” is intended to indicate that the SLGP polypeptide and the non-SLGP polypeptide are fused in-frame to each other. The non-SLGP polypeptide can be fused to the N-terminus or C-terminus of the SLGP polypeptide.

For example, in one embodiment, the fusion protein is a GST-SLGP fusion protein in which the SLGP sequences are fused to the C-terminus of the GST sequences. Such fusion proteins can facilitate the purification of recombinant SLGP. In another embodiment, the fusion protein is a SLGP protein containing a heterologous signal sequence at its N-terminus. For example, a native SLGP signal sequence can be removed and replaced with a signal sequence from another protein. In certain host cells (e.g., mammalian host cells), expression and/or secretion of SLGP can be increased through use of a heterologous signal sequence.

The SLGP fusion proteins of the invention can be incorporated into pharmaceutical compositions and administered to a subject in vivo. The SLGP fusion proteins can be used to affect the bioavailability of a SLGP substrate. Use of SLGP fusion proteins may be useful therapeutically for the treatment of SLGP-related disorders (e.g., paroxysmal nocturnal hemoglobinuria). Moreover, the SLGP-fusion proteins of the invention can be used as immunogens to produce anti-SLGP antibodies in a subject, to purify SLGP ligands and in screening assays to identify molecules which inhibit the interaction of SLGP with a SLGP ligand.

Moreover, the SLGP-fusion proteins of the invention can be used as immunogens to produce anti-SLGP antibodies in a subject, to purify SLGP ligands and in screening assays to identify molecules which inhibit the interaction of SLGP with an SLGP substrate.

Preferably, a SLGP chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). An SLGP-encoding nucleic acid can be cloned into such an expression vector such that the fusion moiety is linked in-frame to the SLGP protein.

The present invention also pertains to variants of the SLGP proteins which function as either SLGP agonists (mimetics) or as SLGP antagonists. Variants of the SLGP proteins can be generated by mutagenesis, e.g., discrete point mutation or truncation of a SLGP protein. An agonist of the SLGP proteins can retain substantially the same, or a subset, of the biological activities of the naturally occurring form of a SLGP protein. An antagonist of a SLGP protein can inhibit one or more of the activities of the naturally occurring form of the SLGP protein by, for example, competitively inhibiting the protease activity of a SLGP protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the SLGP protein.

In one embodiment, variants of a SLGP protein which function as either SLGP agonists (mimetics) or as SLGP antagonists can be identified by screening combinatorial libraries of mutants, e.g., truncation mutants, of a SLGP protein for SLGP protein agonist or antagonist activity. In one embodiment, a variegated library of SLGP variants is generated by combinatorial mutagenesis at the nucleic acid level and is encoded by a variegated gene library. A variegated library of SLGP variants can be produced by, for example, enzymatically ligating a mixture of synthetic oligonucleotides into gene sequences such that a degenerate set of potential SLGP sequences is expressible as individual polypeptides, or alternatively, as a set of larger fusion proteins (e.g., for phage display) containing the set of SLGP sequences therein. There are a variety of methods which can be used to produce libraries of potential SLGP variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential SLGP sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a SLGP protein coding sequence can be used to generate a variegated population of SLGP fragments for screening and subsequent selection of variants of a SLGP protein. In one embodiment, a library of coding sequence fragments can be generated by treating a double stranded PCR fragment of a SLGP coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the SLGP protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of SLGP proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a new technique which enhances the frequency of functional mutants in the libraries, can be used in combination with the screening assays to identify SLGP variants (Arkin and Yourvan (1992) PNAS89:7811-7815; Delgrave et al. (1993) Protein Engineering 6(3):327-331).

In one embodiment, cell based assays can be exploited to analyze a variegated SLGP library. For example, a library of expression vectors can be transfected into a cell line which ordinarily synthesizes SLGP. The transfected cells are then cultured such that a particular mutant SLGP is expressed and the effect of expression of the mutant on SLGP activity in the cell can be detected, e.g., by any of a number of activity assays for native SLGP protein. Plasmid DNA can then be recovered from the cells which score for modulated SLGP activity, and the individual clones further characterized.

An isolated SLGP protein, or a portion or fragment thereof, can be used as an immunogen to generate antibodies that bind SLGP using standard techniques for polyclonal and monoclonal antibody preparation. A full-length SLGP protein can be used or, alternatively, the invention provides antigenic peptide fragments of SLGP for use as immunogens. The antigenic peptide of SLGP comprises at least 8 amino acid residues of the amino acid sequence shown in SEQ ID NO:2 and encompasses an epitope of SLGP such that an antibody raised against the peptide forms a specific immune complex with SLGP. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of SLGP that are located on the surface of the protein, e.g., hydrophilic regions, as well as regions with high antigenicity.

An SLGP immunogen typically is used to prepare antibodies by immunizing a suitable subject, (e.g., rabbit, goat, mouse or other mammal) with the immunogen. An appropriate immunogenic preparation can contain, for example, recombinantly expressed SLGP protein or a chemically synthesized SLGP polypeptide. The preparation can further include an adjuvant, such as Freund's complete or incomplete adjuvant, or similar immunostimulatory agent. Immunization of a suitable subject with an immunogenic SLGP preparation induces a polyclonal anti-SLGP antibody response.

Accordingly, another aspect of the invention pertains to anti-SLGP antibodies. The term “antibody” as used herein refers to immunoglobulin molecules and immunologically active portions of immunoglobulin molecules, i.e., molecules that contain an antigen binding site which specifically binds (immunoreacts with) an antigen, such as SLGP. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind SLGP. The term “monoclonal antibody” or “monoclonal antibody composition”, as used herein, refers to a population of antibody molecules that contain only one species of an antigen binding site capable of immunoreacting with a particular epitope of SLGP. A monoclonal antibody composition thus typically displays a single binding affinity for a particular SLGP protein with which it immunoreacts.

Polyclonal anti-SLGP antibodies can be prepared as described above by immunizing a suitable subject with a SLGP immunogen. The anti-SLGP antibody titer in the immunized subject can be monitored over time by standard techniques, such as with an enzyme linked immunosorbent assay (ELISA) using immobilized SLGP. If desired, the antibody molecules directed against SLGP can be isolated from the mammal (e.g., from the blood) and further purified by well known techniques, such as protein A chromatography to obtain the IgG fraction. At an appropriate time after immunization, e.g., when the anti-SLGP antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem 0.255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a SLGP immunogen as described above, and the culture supernatants of the resulting hybridoma cells are screened to identify a hybridoma producing a monoclonal antibody that binds SLGP.

Any of the many well known protocols used for fusing lymphocytes and immortalized cell lines can be applied for the purpose of generating an anti-SLGP monoclonal antibody (see, e.g., G. Galfre et al. (1977) Nature 266:55052; Gefter et al. Somatic Cell Genet., cited supra; Lemer, Yale J. Biol. Med., cited supra; Kenneth, Monoclonal Antibodies, cited supra). Moreover, the ordinarily skilled worker will appreciate that there are-many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Agl4 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind SLGP, e.g., using a standard ELISA assay.

Alternative to preparing monoclonal antibody-secreting hybridomas, a monoclonal anti-SLGP antibody can be identified and isolated by screening a recombinant combinatorial immunoglobulin library (e.g., an antibody phage display library) with SLGP to thereby isolate immunoglobulin library members that bind SLGP. Kits for generating and screening phage display libraries are commercially available (e.g., the Pharmacia Recombinant Phage Antibody System, Catalog No. 27-9400-01; and the Stratagene SurZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc. Acid Res. 19:4133-4137; Barbas et al. (1991) PNAS 88:7978-7982; and McCafferty et al. Nature (1990) 348:552-554.

Additionally, recombinant anti-SLGP antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-SLGP antibody (e.g., monoclonal antibody) can be used to isolate SLGP by standard techniques, such as affinity chromatography or immunoprecipitation. An anti-SLGP antibody can facilitate the purification of natural SLGP from cells and of recombinantly produced SLGP expressed in host cells. Moreover, an anti-SLGP antibody can be used to detect SLGP protein (e.g., in a cellular lysate or cell supernatant) in order to evaluate the abundance and pattern of expression of the SLGP protein. Anti-SLGP antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein,-dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Furthermore, given the fact that an important use for the SLGP molecules of the present invention is in the screening for SLGP ligands (e.g., surrogate ligands) and/or SLGP modulators, it is intended that the following are also within the scope of the present invention: “isolated” or “purified” SLGP ligands or SLGP modulators, biologically-active portions of SLGP ligands or SLGP modulators, chimeric or fusion proteins comprising all or a portion of a SLGP ligand or SLGP modulator, and antibodies comprising all or a portion of a SLGP ligand or SLGP modulator.

III. Recombinant Expression Vectors and Host Cells

Another aspect of the invention pertains to vectors, preferably expression vectors, containing a nucleic acid encoding a SLGP protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcriptibn/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., SLGP proteins, mutant forms of SLGP proteins, fusion proteins, etc.).

The recombinant expression vectors of the invention can be designed for expression of SLGP proteins in prokaryotic or eukaryotic cells. For example, SLGP proteins can be expressed in bacterial cells such as E. coli , insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in SLGP activity assays, (e.g., direct assays or competitive assays described in detail below), or to generate antibodies specific for SLGP proteins, for example. In a preferred embodiment, a SLGP fusion protein expressed in a retroviral expression vector of the present invention can be utilized to infect bone marrow cells which are subsequently transplanted into irradiated recipients. The pathology of the subject recipient is then examined after sufficient time has passed (e.g six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the SLGP expression vector is a yeast expression vector. Examples of vectors for expression in yeast S. cerivisae include pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, SLGP proteins can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed, Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to SLGP mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which an SLGP nucleic acid molecule of the invention is introduced, e.g., an SLGP nucleic acid molecule within a recombinant expression vector or an SLGP nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a SLGP protein can be expressed in bacterial cells such as E. coli , insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed, Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a SLGP protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a SLGP protein. Accordingly, the invention further provides methods for producing a SLGP protein using the host cells of the invention. In one embodiment, the method comprises culturing the host cell of invention (into which a recombinant expression vector encoding a SLGP protein has been introduced) in a suitable medium such that a SLGP protein is produced. In another embodiment, the method further comprises isolating a SLGP protein from the medium or the host cell.

The host cells of the invention can also be used to produce nonhuman transgenic animals. For example, in one embodiment, a host cell of the invention is a fertilized oocyte or an embryonic stem cell into which SLGP-coding sequences have been introduced. Such host cells can then be used to create non-human transgenic animals in which exogenous SLGP sequences have been introduced into their genome or homologous recombinant animals in which endogenous SLGP sequences have been altered. Such animals are useful for studying the function and/or activity of a SLGP and for identifying and/or evaluating modulators of SLGP activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous SLGP gene has been altered by homologous recombination between the endogenous gene and an exogenous DNA molecule introduced into a cell of the animal, e.g., an embryonic cell of the animal, prior to development of the animal.

A transgenic animal of the invention can be created by introducing an SLGP-encoding nucleic acid into the male pronuclei of a fertilized oocyte, e.g., by microinjection, retroviral infection, and allowing the oocyte to develop in a pseudopregnant female foster animal. The SLGP cDNA sequence of SEQ ID NO:1 can be introduced as a transgene into the genome of a non-human animal. Alternatively, a nonhuman homologue of a human SLGP gene, such as a mouse or rat SLGP gene, can be used as a transgene. Alternatively, an SLGP gene homologue, such as another GPCR family member, can be isolated based on hybridization to the SLGP cDNA sequences of SEQ ID NO:1, SEQ ID NO:3, or the DNA insert of the plasmid deposited with ATCC as Accession Number ______ (described further in subsection I above) and used as a transgene. Intronic sequences and polyadenylation signals can also be included in the transgene to increase the efficiency of expression of the transgene. A tissue-specific regulatory sequence(s) can be operably linked to an SLGP transgene to direct expression of an SLGP protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of an SLGP transgene in its genome and/or expression of SLGP mRNA in tissues or cells of the animals. A transgenic founder animal can then be used to breed additional animals carrying the transgene. Moreover, transgenic animals carrying a transgene encoding an SLGP protein can further be bred to other transgenic animals carrying other transgenes.

To create a homologous recombinant animal, a vector is prepared which contains at least a portion of an SLGP gene into which a deletion, addition or substitution has been introduced to thereby alter, e.g., functionally disrupt, the SLGP gene. The SLGP gene can be a human gene (e.g., the cDNA of SEQ ID NO:3), but more preferably, is a non-human homologue of a human SLGP gene (e.g., a cDNA isolated by stringent hybridization with the nucleotide sequence of SEQ ID NO:1). For example, a mouse SLGP gene can be used to construct a homologous recombination nucleic acid molecule, e.g., a vector, suitable for altering an endogenous SLGP gene in the mouse genome. In a preferred embodiment, the homologous recombination nucleic acid molecule is designed such that, upon homologous recombination, the endogenous SLGP gene is functionally disrupted (i.e., no longer encodes a functional protein; also referred to as a “knock out” vector). Alternatively, the homologous recombination nucleic acid molecule can be designed such that, upon homologous recombination, the endogenous SLGP gene is mutated or otherwise altered but still encodes functional protein (e.g., the upstream regulatory region can be altered to thereby alter the expression of the endogenous SLGP protein). In the homologous recombination nucleic acid molecule, the altered portion of the SLGP gene is flanked at its 5′ and 3′ ends by additional nucleic acid sequence of the SLGP gene to allow for homologous recombination to occur between the exogenous SLGP gene carried by the homologous recombination nucleic acid molecule and an endogenous SLGP gene in a cell, e.g., an embryonic stem cell. The additional flanking SLGP nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced SLGP gene has homologously recombined with the endogenous SLGP gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The recontructed oocyte is then cultured such that it develops to morula or blastocyte and then transferred to pseudopregnant female foster animal. The offspring borne of this female foster animal will be a clone of the animal from which the cell, e.g., the somatic cell, is isolated. Alternatively, a cell, e.g., an embryonic stem cell, from the inner cell mass of a developing embryo can be transformed with a preferred transgene. Alternatively, a cell, e.g., a somatic cell, from cell culture line can be transformed with a preferred transgene and induced to exit the growth cycle and enter Go phase. The cell can then be fused, e.g., through the use of electrical pulses, to an enucleated mammalian oocyte. The reconstructed oocyte is then cultured such that it develops to morula or blastocyst and then transferred to pseudopregnant female foster animal. The offspring borne of this female foster animal will be a clone of the animal from which the nuclear donor cell, e.g., the somatic cell, is isolated.

IV. Pharmaceutical Compositions

The SLGP nucleic acid molecules, SLGP proteins, anti-SLGP antibodies, SLGP ligands, and SLGP modulators (also referred to herein as “active compounds”) of the invention can be incorporated into pharmaceutical compositions suitable for administration. Such compositions typically comprise the nucleic acid molecule, protein, antibody, or modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a SLGP protein or anti-SLGP antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). As described herein, a SLGP protein of the invention has one or more of the following activities: (i) interaction of a SLGP protein with soluble SLGP ligand (e.g., CD55); (ii) interaction of a SLGP protein with a membrane-bound non-SLGP protein; (iii) interaction of a SLGP protein with an intracellular protein (e.g., an intracellular enzyme or signal transduction molecule); and (iv) indirect interaction of a SLGP protein with an intracellular protein (e.g., a downstream signal transduction molecule), and can can thus be used in, for example, (1) modulation of cellular signal transduction, either in vitro or in vivo; (2) regulation of activation in a cell expressing a SLGP protein (e.g., leukocyte activation); (3) regulation of a hematopoietic cell expressing a SLGP protein, wherein said hematopoietic cell is involved in inflammation; (4) regulation of small synaptic vesicle exocytosis (e.g., small synaptic vesicle exocytosis in neurons in response to exposure to alpha-latrotoxin); (5) regulation of inflammation. The isolated nucleic acid molecules of the invention can be used, for example, to express SLGP protein (e.g., via a recombinant expression vector in a host cell in gene therapy applications), to detect SLGP mRNA (e.g., in a biological sample) or a genetic alteration in a SLGP gene, and to modulate SLGP activity, as described further below. The SLGP proteins can be used to treat disorders characterized by insufficient or excessive production of a SLGP protein and/or SLGP ligand. In addition, the SLGP proteins can be used to screen drugs or compounds which modulate the SLGP activity as well as to treat disorders characterized by insufficient or excessive production of SLGP protein or production of SLGP protein forms which have decreased or aberrant activity compared to SLGP wild type protein. Moreover, the anti-SLGP antibodies of the invention can be used to detect and isolate SLGP proteins, regulate the bioavailability of SLGP proteins, and modulate SLGP activity.

A. Screening Assays:

The invention provides a method (also referred to herein as a “screening assay”) for identifying modulators, i.e., candidate or test compounds or agents (e.g., peptides, peptidomimetics, small molecules or other drugs) which bind to SLGP proteins, or have a stimulatory or inhibitory effect on, for example, SLGP expression or SLGP activity.

In one embodiment, the invention provides assays for screening candidate or test compounds which bind to or modulate the activity of a SLGP protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a SLGP protein on the cell surface is contacted with a test compound and the ability of the test compound to bind to the SLGP protein determined. The cell, for example, can be of mammalian origin or a yeast cell. Determining the ability of the test compound to bind to a SLGP protein can be accomplished, for example, by coupling the test compound with a radioisotope or enzymatic label such that binding of the test compound to the SLGP protein can be determined by detecting the labeled compound in a complex. For example, test compounds can be labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a test compound to interact with a SLGP protein without the labeling of any of the interactants. For example, a microphysiometer can be used to detect the interaction of a test compound with a SLGP protein without the labeling of either the test compound or the receptor. McConnell, H. M. et al. (1992) Science 257:1906-1912. As used herein, a “microphysiometer” (e.g., Cytosensorm) is an analytical instrument that measures the rate at which a cell acidifies its environment using a light-addressable potentiometric sensor (LAPS). Changes in this acidification rate can be used as an indicator of the interaction between ligand and receptor.

In a preferred embodiment, the assay comprises contacting a cell which expresses a SLGP protein or biologically active portion thereof, on the cell surface with a SLGP ligand, to form an assay mixture, contacting the assay mixture with a test compound, and determining the ability of the test compound to interact with the SLGP protein or biologically active portion thereof, wherein determining the ability of the test compound to interact with the SLGP protein or biologically active portion thereof, comprises determining the ability of the test compound to preferentially bind to the SLGP protein or biologically active portion thereof, as compared to the ability of the SLGP ligand to bind to the SLGP protein or biologically active portion thereof.

Determining the ability of the SLGP ligand or SLGP modulator to bind to or interact with a SLGP protein or biologically active portion thereof, can be accomplished by one of the methods described above for determining direct binding. In a preferred embodiment, determining the ability of the SLGP ligand or modulator to bind to or interact with a SLGP protein or biologically active portion thereof, can be accomplished by determining the activity of a SLGP protein or of a downstream SLGP target molecule. For example, the target molecule can be a cellular second messenger, and the activity of the target molecule can be determined by detecting induction of of the target (i.e. intracellular Ca²⁺, diacylglycerol, IP3, etc.), detecting catalytic/enzymatic activity of the target on an appropriate substrate, detecting the induction of a reporter gene (comprising a SLGP-responsive regulatory element operatively linked to a nucleic acid encoding a detectable marker, e.g., luciferase), or detecting a cellular response, for example, a proliferative response or an inflammatory response. Accordingly, in one embodiment the present invention involves a method of identifying a compound which modulates the activity of a SLGP protein, comprising contacting a cell which expresses a SLGP protein with a test compound, determining the ability of the test compound to modulate the activity the SLGP protein, and identifying the compound as a modulator of SLGP activity. In another embodiment, the present invention involves a method of identifying a compound which modulates the activity of a SLGP protein, comprising contacting a cell which expresses a SLGP protein with a test compound, determining the ability of the test compound to modulate the activity of a downstream SLGP target molecule, and identifying the compound as a modulator of SLGP activity.

In yet another embodiment, an assay of the present invention is a cell-free assay in which a SLGP protein or biologically active portion thereof is contacted with a test compound and the ability of the test compound to bind to the SLGP protein or biologically active portion thereof is determined. Binding of the test compound to the SLGP protein can be determined either directly or indirectly as described above. Binding of the test compound to the SLGP protein can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore™). Changes in the optical phenomenon of surface plasmon resonance (SPR) can be used as an indication of real-time reactions between biological molecules.

In a preferred embodiment, the assay includes contacting the SLGP protein or biologically active portion thereof with a known ligand which binds SLGP to form an assay mixture, contacting the assay mixture with a test compound, and determining the ability of the test compound to interact with a SLGP protein, wherein determining the ability of the test compound to interact with a SLGP protein comprises determining the ability of the test compound to preferentially bind to SLGP or biologically active portion thereof as compared to the known ligand.

In another embodiment, the assay is a cell-free assay in which a SLGP protein or biologically active portion thereof is contacted with a test compound and the ability of the test compound to modulate (e.g., stimulate or inhibit) the activity of the SLGP protein or biologically active portion thereof is determined. Determining the ability of the test compound to modulate the activity of a SLGP protein can be accomplished, for example, by determining the ability of the SLGP protein to modulate the activity of a downstream SLGP target molecule by one of the methods described above for cell-based assays. For example, the catalytic/enzymatic activity of the target molecule on an appropriate substrate can be determined as previously described.

In yet another embodiment, the cell-free assay involves contacting a SLGP protein or biologically active portion thereof with a known ligand which binds the SLGP protein to form an assay mixture, contacting the assay mixture with a test compound, and determining the ability of the test compound to interact with the SLGP protein, wherein determining the ability of the test compound to interact with the SLGP protein comprises determining the ability of the test compound to preferentially bind to or modulate the activity of a SLGP target molecule, as compared to the known ligand.

The cell-free assays of the present invention are amenable to use of both soluble and/or membrane-bound forms of isolated proteins (e.g. SLGP proteins or biologically active portions thereof or SLGP proteins). In the case of cell-free assays in which a membrane-bound form an isolated protein is used (e.g., a SLGP protein) it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit® R, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either SLGP or its target molecule to facilitate separation of complexed from uncomplexed forms of one or both of the proteins, as well as to accommodate automation of the assay. Binding of a test compound to a SLGP protein, or interaction of a SLGP protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/SLGP fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or SLGP protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of SLGP binding or activity determined using standard techniques.

Other techniques for immobilizing proteins on matrices can also be used in the screening assays of the invention. For example, either a SLGP protein or a SLGP target molecule can be immobilized utilizing conjugation of biotin and streptavidin. Biotinylated SLGP protein or target molecules can be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with SLGP protein or target molecules but which do not interfere with binding of the SLGP protein to its target molecule can be derivatized to the wells of the plate, and unbound target or SLGP protein trapped in the wells by antibody conjugation. Methods for detecting such complexes, in addition to those described above for the GST-immobilized complexes, include immunodetection of complexes using antibodies reactive with the SLGP protein or target molecule, as well as enzyme-linked assays which rely on detecting an enzymatic activity associated with the SLGP protein or target molecule.

In another embodiment, modulators of SLGP expression are identified in a method wherein a cell is contacted with a candidate compound and the expression of SLGP mRNA or protein in the cell is determined. The level of expression of SLGP mRNA or protein in the presence of the candidate compound is compared to the level of expression of SLGP mRNA or protein in the absence of the candidate compound. The candidate compound can then be identified as a modulator of SLGP expression based on this comparison. For example, when expression of SLGP mRNA or protein is greater (statistically significantly greater) in the presence of the candidate compound than in its absence, the candidate compound is identified as a stimulator of SLGP mRNA or protein expression. Alternatively, when expression of SLGP mRNA or protein is less (statistically significantly less) in the presence of the candidate compound than in its absence, the candidate compound is identified as an inhibitor of SLGP mRNA or protein expression. The level of SLGP mRNA or protein expression in the cells can be determined by methods described herein for detecting SLGP mRNA or protein.

In yet another aspect of the invention, the SLGP proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with SLGP (“SLGP-binding proteins” or “SLGP-bp”) and are involved in SLGP activity. Such SLGP-binding proteins are also likely to be involved in the propagation of signals by the SLGP proteins as, for example, downstream elements of a SLGP-mediated signaling pathway. Alternatively, such SLGP-binding proteins are likely to be cell-surface molecules associated with non-SLGP expressing cells, wherein such SLGP-binding proteins are involved in chemoattraction.

The two-hybrid system is based on the modular nature of most transcription factors, which consist of separable DNA-binding and activation domains. Briefly, the assay utilizes two different DNA constructs. In one construct, the gene that codes for a SLGP protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a SLGP-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the SLGP protein.

This invention further pertains to novel agents identified by the above-described screening assays. Accordingly, it is within the scope of this invention to further use an agent identified as described herein in an appropriate animal model. For example, an agent identified as described herein (e.g., a SLGP modulating agent, an antisense SLGP nucleic acid molecule, a SLGP-specific antibody, or a SLGP-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the SLGP nucleotide sequences, described herein, can be used to map the location of the SLGP genes on a chromosome. The mapping of the SLGP sequences to chromosomes is an important first step in correlating these sequences with genes associated with disease.

Briefly, SLGP genes can be mapped to chromosomes by preparing PCR primers (preferably 15-25 bp in length) from the SLGP nucleotide sequences. Computer analysis of the SLGP sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the SLGP sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the SLGP nucleotide sequences to design oligonucleotide primers, sublocalization can be achieved with panels of fragments from specific chromosomes. Other mapping strategies which can similarly be used to map a 9o, 1p, or lv sequence to its chromosome include in situ hybridization (described in Fan, Y. et al. (1990) PNAS, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the SLGP gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The SLGP sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the SLGP nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The SLGP nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1, can comfortably provide positive individual identification with a panel of perhaps 10 to 1,000 primers which each yield a noncoding amplified sequence of 100 bases. If predicted coding sequences, such as those in SEQ ID NO:3 are used, a more appropriate number of primers for positive individual identification would be 500-2,000.

If a panel of reagents from SLGP nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of Partial SLGP Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1 are particularly appropriate for this use as greater numbers of polymorphisms occur in the noncoding regions, making it easier to differentiate individuals using this technique. Examples of polynucleotide reagents include the SLGP nucleotide sequences or portions thereof, e.g., fragments derived from the noncoding regions of SEQ ID NO:1, having a length of at least 20 bases, preferably at least 30 bases.

The SLGP nucleotide sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such SLGP probes can be used to identify tissue by species and/or by organ type.

In a similar fashion, these reagents, e.g., SLGP primers or probes can be used to screen tissue culture for contamination (i.e. screen for the presence of a mixture of different types of cells in a culture).

C. Predictive Medicine:

The present invention also pertains to the field of predictive medicine in which diagnostic assays, prognostic assays, and monitoring clinical trails are used for prognostic (predictive) purposes to thereby treat an individual prophylactically. Accordingly, one aspect of the present invention relates to diagnostic assays for determining SLGP protein and/or nucleic acid expression as well as SLGP activity, in the context of a biological sample (e.g., blood, serum, cells, tissue) to thereby determine whether an individual is afflicted with a disease or disorder, or is at risk of developing a disorder, associated with aberrant SLGP expression or activity. The invention also provides for prognostic (or predictive) assays for determining whether an individual is at risk of developing a disorder associated with SLGP protein, nucleic acid expression or activity. For example, mutations in a SLGP gene can be assayed in a biological sample. Such assays can be used for prognostic or predictive purpose to thereby phophylactically treat an individual prior to the onset of a disorder characterized by or associated with SLGP protein, nucleic acid expression or activity.

Another aspect of the invention pertains to monitoring the influence of agents (e.g., drugs, compounds) on the expression or activity of SLGP in clinical trials.

These and other agents are described in further detail in the following sections.

1. Diagnostic Assays

An exemplary method for detecting the presence or absence of SLGP protein or nucleic acid in a biological sample involves obtaining a biological sample from a test subject and contacting the biological sample with a compound or an agent capable of detecting SLGP protein or nucleic acid (e.g., mRNA, genomic DNA) that encodes SLGP protein such that the presence of SLGP protein or nucleic acid is detected in the biological sample. A preferred agent for detecting SLGP mRNA or genomic DNA is a labeled nucleic acid probe capable of hybridizing to SLGP mRNA or genomic DNA. The nucleic acid probe can be, for example, a full-length SLGP nucleic acid, such as the nucleic acid of SEQ ID NO: 1, or a fragment or portion of a SLGP nucleic acid such as an oligonucleotide of at least 15, 30, 50, 100, 250 or 500 nucleotides in length and sufficient to specifically hybridize under stringent conditions to SLGP mRNA or genomic DNA. Other suitable probes for use in the diagnostic assays of the invention are described herein.

A preferred agent for detecting SLGP protein is an antibody capable of binding to SLGP protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect SLGP mRNA, protein, or genomic DNA in a biological sample in vitro as well as in vivo. For example, in vitro techniques for detection of SLGP mRNA include Northern hybridizations and in situ hybridizations. In vitro techniques for detection of SLGP protein include enzyme linked immunosorbent assays (ELISAs), Western blots, immunoprecipitations and immunofluorescence. In vitro techniques for detection of SLGP genomic DNA include Southern hybridizations. Furthermore, in vivo techniques for detection of SLGP protein include introducing into a subject a labeled anti-SLGP antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting SLGP protein, mRNA, or genomic DNA, such that the presence of SLGP protein, mRNA or genomic DNA is detected in the biological sample, and comparing the presence of SLGP protein, mRNA or genomic DNA in the control sample with the presence of SLGP protein, mRNA or genomic DNA in the test sample.

The invention also encompasses kits for detecting the presence of SLGP in a biological sample. For example, the kit can comprise a labeled compound or agent capable of detecting SLGP protein or mRNA in a biological sample; means for determining the amount of SLGP in the sample; and means for comparing the amount of SLGP in the sample with a standard. The compound or agent can be packaged in a suitable container. The kit can further comprise instructions for using the kit to detect SLGP protein or nucleic acid.

2. Prognostic Assays

The diagnostic methods described herein can furthermore be utilized to identify subjects having or at risk of developing a disease or disorder associated with aberrant SLGP expression or activity. As used herein, the term “aberrant” includes an SLGP expression or activity which deviates from the wild type SLGP expression or activity. Aberrant expression or activity includes increased or decreased expression or activity, as well as expression or activity which does not follow the wild type developmental pattern of expression or the subcellular pattern of expression. For example, aberrant SLGP expression or activity is intended to include the cases in which a mutation in the SLGP gene causes the SLGP gene to be under-expressed or over-expressed and situations in which such mutations result in a non-functional SLGP protein or a protein which does not function in a wild-type fashion, e.g., a protein which does not interact with an SLGP ligand or one which interacts with a non-SLGP ligand.

The assays described herein, such as the preceding diagnostic assays or the following assays, can be utilized to identify subjects having or at risk of developing a disease or disorder associated with aberrant SLGP expression or activity. For example, the assays described herein, such as the preceding diagnostic assays or the following assays, can be utilized to identify a subject having or at risk of developing a disorder associated with SLGP protein, nucleic acid expression or activity such as an inflammatory disorder. Alternatively, the prognostic assays can be utilized to identify a subject having or at risk for developing an inflammatory disorder. Thus, the present invention provides a method for identifying a disease or disorder associated with aberrant SLGP expression or activity in which a test sample is obtained from a subject and SLGP protein or nucleic acid (e.g, mRNA, genomic DNA) is detected, wherein the presence of SLGP protein or nucleic acid is diagnostic for a subject having or at risk of developing a disease or disorder associated with aberrant SLGP expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant SLGP expression or activity. For example, such methods can be used to determine whether a subject can be effectively treated with an agent for a disorder, such as an inflammatory disorder. Alternatively, such methods can be used to determine whether a subject can be effectively treated with an agent for an inflammatory disease. Thus, the present invention provides methods for determining whether a subject can be effectively treated with an agent for a disorder associated with aberrant SLGP expression or activity in which a test sample is obtained and SLGP protein or nucleic acid expression or activity is detected (e.g., wherein the abundance of SLGP protein or nucleic acid expression or activity is diagnostic for a subject that can be administered the agent to treat a disorder associated with aberrant SLGP expression or activity.)

The methods of the invention can also be used to detect genetic alterations in a SLGP gene, thereby determining if a subject with the altered gene is at risk for a disorder characterized by an aberrant inflammatory response. In preferred embodiments, the methods include detecting, in a sample of cells from the subject, the presence or absence of a genetic alteration characterized by at least one of an alteration affecting the integrity of a gene encoding a SLGP-protein, or the mis-expression of the SLGP gene. For example, such genetic alterations can be detected by ascertaining the existence of at least one of 1) a deletion of one or more nucleotides from a SLGP gene; 2) an addition of one or more nucleotides to a SLGP gene; 3) a substitution of one or more nucleotides of a SLGP gene, 4) a chromosomal rearrangement of a SLGP gene; 5) an alteration in the level of a messenger RNA transcript of a SLGP gene, 6) aberrant modification of a SLGP gene, such as of the methylation pattern of the genomic DNA, 7) the presence of a non-wild type splicing pattern of a messenger RNA transcript of a SLGP gene, 8) a non-wild type level of a SLGP-protein, 9) allelic loss of a SLGP gene, and 10) inappropriate post-translational modification of a SLGP-protein. As described herein, there are a large number of assay techniques known in the art which can be used for detecting alterations in a SLGP gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the SLGP-gene (see Abravaya et al. (1995) Nucleic Acids Res 0.23:675-682). This method can include the steps of collecting a sample of cells from a patient, isolating nucleic acid (e.g., genomic, mRNA or both) from the cells of the sample, contacting the nucleic acid sample with one or more primers which specifically hybridize to a SLGP gene under conditions such that hybridization and amplification of the SLGP-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et all, 1988, Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a SLGP gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in SLGP can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in SLGP can be identified in two dimensional arrays containing light-generated DNA probes as described in Cronin, M. T. et al. supra Briefly, a first hybridization array of probes can be used to scan through long stretches of DNA in a sample and control to identify base changes between the sequences by making linear arrays of sequential ovelapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the SLGP gene and detect mutations by comparing the sequence of the sample SLGP with the corresponding wild-type (control) sequence. Examples of sequencing reactions include those based on techniques developed by Maxim and Gilbert ((1977) PNAS 74:560) or Sanger ((1977) PNAS 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the SLGP gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type SLGP sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad Sci USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in SLGP cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a SLGP sequence, e.g., a wild-type SLGP sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in SLGP genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat Res 285:125-144; and Hayashi (1992) Genet Anal Tech Appl 9:73-79). Single-stranded DNA fragments of sample and control SLGP nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method-utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a SLGP gene.

Furthermore, any cell type or tissue in which SLGP is expressed may be utilized in the prognostic assays described herein.

3. Monitoring of Effects During Clinical Trials

Monitoring the influence of agents (e.g., drugs, compounds) on the expression or activity of a SLGP protein (e.g., modulation of an inflammatory responese) an be applied not only in basic drug screening, but also in clinical trials. For example, the effectiveness of an agent determined by a screening assay as described herein to increase SLGP gene expression, protein levels, or upregulate SLGP activity, can be monitored in clinical trails of subjects exhibiting decreased SLGP gene expression, protein levels, or downregulated SLGP activity. Alternatively, the effectiveness of an agent determined by a screening assay to decrease SLGP gene expression, protein levels, or downregulate SLGP activity, can be monitored in clinical trails of subjects exhibiting increased SLGP gene expression, protein levels, or upregulated SLGP activity. In such clinical trials, the expression or activity of a SLGP gene, and preferably, other genes that have been implicated in, for example, an inflammatory disorder can be used as a “read out” or markers of the phenotype of a particular cell.

For example, and not by way of limitation, genes, including SLGP, that are modulated in cells by treatment with an agent (e.g., compound, drug or small molecule) which modulates SLGP activity (e.g., identified in a screening assay as described herein) can be identified. Thus, to study the effect of agents on inflammatory disorders, for example, in a clinical trial, cells can be isolated and RNA prepared and analyzed for the levels of expression of SLGP and other genes implicated in the inflammatory disorder, respectively. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of SLGP or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression of a SLGP protein, mRNA, or genomic DNA in the preadministration sample; (iii) obtaining one or more post-administration samples from the subject; (iv) detecting the level of expression or activity of the SLGP protein, mRNA, or genomic DNA in the post-administration samples; (v) comparing the level of expression or activity of the SLGP protein, mRNA, or genomic DNA in the pre-administration sample with the SLGP protein, mRNA, or genomic DNA in the post administration sample or samples; and (vi) altering the administration of the agent to the subject accordingly. For example, increased administration of the agent may be desirable to increase the expression or activity of SLGP to higher levels than detected, i.e., to increase the effectiveness of the agent. Alternatively, decreased administration of the agent may be desirable to decrease expression or activity of SLGP to lower levels than detected, i.e. to decrease the effectiveness of the agent. According to such an embodiment, SLGP expression or activity may be used as an indicator of the effectiveness of an agent, even in the absence of an observable phenotypic response.

C. Methods of Treatment:

The present invention provides for both prophylactic and therapeutic methods of treating a subject at risk of (or susceptible to) a disorder or having a disorder associated with aberrant SLGP expression or activity. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the SLGP molecules of the present invention or SLGP modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant SLGP expression or activity, by administering to the subject a SLGP or an agent which modulates SLGP expression or at least one SLGP activity. Subjects at risk for a disease which is caused or contributed to by aberrant SLGP expression or activity can be identified by, for example, any or a combination of diagnostic or prognostic assays as described herein. Administration of a prophylactic agent can occur prior to the manifestation of symptoms characteristic of the SLGP aberrancy, such that a disease or disorder is prevented or, alternatively, delayed in its progression. Depending on the type of SLGP aberrancy, for example, a SLGP, SLGP agonist or SLGP antagonist agent can be used for treating the subject. The appropriate agent can be determined based on screening assays described herein. The prophylactic methods of the present invention are further discussed in the following subsections.

2. Therapeutic Methods

Another aspect of the invention pertains to methods of modulating SLGP expression or activity for therapeutic purposes. Accordingly, in an exemplary embodiment, the modulatory method of the invention involves contacting a cell with a SLGP molecule of the present invention such that the activity of a SLGP is modulated. Alternatively, the modulatory method of the invention involves contacting a cell with an agent that modulates one or more of the activities of SLGP protein activity associated with the cell. An agent that modulates SLGP protein activity can be an agent as described herein, such as a nucleic acid or a protein, a naturally-occurring target molecule of a SLGP protein (e.g., CD55), a SLGP antibody, a SLGP agonist or antagonist, a peptidomimetic of a SLGP agonist or antagonist, or other small molecule. In one embodiment, the agent stimulates one or more SLGP activites. Examples of such stimulatory agents include active SLGP protein and a nucleic acid molecule encoding SLGP that has been introduced into the cell. In another embodiment, the agent inhibits one or more SLGP activites. Examples of such inhibitory agents include antisense SLGP nucleic acid molecules and anti-SLGP antibodies. These modulatory methods can be performed in vitro (e.g., by culturing the cell with the agent) or, alternatively, in vivo (e.g, by administering the agent to a subject). As such, the present invention provides methods of treating an individual afflicted with a disease or disorder characterized by aberrant expression or activity of a SLGP protein or nucleic acid molecule. In one embodiment, the method involves administering an agent (e.g., an agent identified by a screening assay described herein), or combination of agents that modulates (e.g., upregulates or downregulates) SLGP expression or activity. In another embodiment, the method involves administering a SLGP protein or nucleic acid molecule as therapy to compensate for reduced or aberrant SLGP expression or activity.

Stimulation of SLGP activity is desirable in situations in which SLGP is abnormally downregulated and/or in which increased SLGP activity is likely to have a beneficial effect. Likewise, inhibition of SLGP activity is desirable in situations in which SLGP is abnormally upregulated and/or in which decreased SLGP activity is likely to have a beneficial effect (e.g., inflammation).

3. Pharmacogenomics

The SLGP molecules of the present invention, as well as agents, or modulators which have a stimulatory or inhibitory effect on SLGP activity (e.g., SLGP gene expression) as identified by a screening assay described herein can be administered to individuals to treat (prophylactically or therapeutically) disorders (e.g, inflammatory disorders) associated with aberrant SLGP activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a SLGP molecule or SLGP modulator as well as tailoring the dosage and/or therapeutic regimen of treatment with a SLGP molecule or SLGP modulator.

Pharmacogenomics deals with clinically significant hereditary variations in the response to drugs due to altered drug disposition and abnormal action in affected persons. See e.g., Eichelbaum, M., Clin Exp Pharmacol Physiol, 1996, 23(10-11):983-985 and Linder, M. W., Clin Chem, 1997, 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava bearis.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a SLGP protein or SLGP protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a SLGP molecule or SLGP modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a SLGP molecule or SLGP modulator, such as a modulator identified by one of the exemplary screening assays described herein.

This invention is further illustrated by the following examples which should not be construed as limiting. The contents of all references, patents and published patent applications cited throughout this application are hereby incorporated by reference. References throughout the instant specification to websites maintained as part of the World Wide Web are referred to herein by the prefix http://. The information contained in such websites is publically-availlable and can be accessed elctronically by contacting the cited address.

### EXAMPLES

### Example 1

### Identification And Characterization of SLGP cDNAs

In this example, the identification and characterization of the gene encoding human SLGP (also referred to as “Fchrb021h09”) is described.

Isolation of the Human SLGP cDNA

In order to identify novel secreted and/or membrane-bound proteins, a program termed ‘signal sequence trapping’ was utilized to analyse the sequences of several cDNAs of a cDNA library derived from bronchial epithelial cells which had been stimulated with the cytokine, TNFα. This analysis identified a human clone having an insert of approximately 3 kb containing a protein-encoding sequence of approximately 2987 nucleotides capable of encoding approximately 690 amino acids of SLGP (e.g., the start met through residue 690 of, for example, SEQ ID NO:2).

The nucleotide sequence encoding the human SLGP protein is shown in FIG. 1 and is set forth as SEQ ID NO:1. The full length protein encoded by this nucleic acid is comprised of about 690 amino acids and has the amino acid sequence shown in FIG. 1 and set forth as SEQ ID NO:2. The coding portion (open reading frame) of SEQ ID NO:1 is set forth as SEQ ID NO:3.

Analysis of Human SLGP

A BLAST search (Altschul et al. (1990) J. Mol. Biol. 215:403) of the nucleotide sequence of human SLGP has revealed that SLGP is significantly similar to a protein identified as human CD 97 (Accession No. U76764) and to a protein identified as rat latrophilin (Acession No.s U78105, U72487).

The SLGP proteins of the present invention contain a siginificant number of structural characteristics of the GPCR family. For instance, the SLGPs of the present invention contain conserved cysteines found in the first 2 extracellular loops (prior to the third and fifth transmembrane domains) of most GPCRs (cys490 and cys602 of SEQ ID NO:2). A highly conserved asparagine residue is present (asn125 in SEQ ID NO:2). SLGP proteins contains a highly conserved leucine (leu154 of SEQ ID NO:2). The two cysteine residues are believed to form a disulfide bond that stabilizes the functional protein structure. A highly conserved asparagine and arginine in the fourth transmembrane domain of the SLGP proteins is present (asp 158 and arg218 of SEQ ID NO:2). The third cytoplasmic loop contains 18 amino acid residues and is thus the longest cytoplasmic loop of the three, characteristic of G protein coupled receptors. Moreover, a highly conserved proline is present (pro307 of SEQ ID NO:2). Proline residues in the fourth, fifth, sixth, and seventh transmembrane domains are thought to introduce kinks in the alpha-helices and may be important in the formation of the ligand binding pocket. Moreover, a conserved tyrosine is present in the seventh transmembrane domain of SLGP-2 (tyr646 of SEQ ID NO:2).

As such, the SLGP family of proteins, like the Secretin family of proteins, are refered to herein as G protein-coupled receptor-like proteins.

SLGP is predicted to contain the following sites: N-glycosylation site at aa 15-18 (NCSY), aa 21-24 (NCTK), aa 64-67 (NLTQ), aa 74-77 (NCTN), aa 127-130 (NKTL), aa 177-180 (NNTI), aa 188-191 (NSTL), aa 249-252 (NSTD), aa 381-384 (NGSW), and at aa 395-398 (NETH); Glycosaminoglycan attachment site at aa 49-52 (SGNG); cAMP- and cGMP-dependent protein kinase phosphorylation sites at aa 360-363 (RKVT); Protein kinase C phosphorylation sites at aa 135-137 (SIK), aa 181-183 (SAK), aa 233-235 (TLR), aa 358-360 (SHR), aa 363-365 (TDR), aa 400-402 (SCR), aa 457-459 (STR), aa 485-487 (TNK), and at aa 558-560 (TTK), aa 667-669 (SRK); Casein kinase II phosphorylation sites at aa 54-57 (TICE), aa 68-71 (SCGE), aa 76-79 (TNTE), aa 94-97 (SNQD), aa 135-138 (SIKE), aa 150-153 (SVTD), aa 155-158 (SPTD), aa 161-164 (TYIE), aa 181-184 (SAKD), aa 190-193 (TLTE), aa 244-247 (TEFD), aa 310-313 (SSSD), aa 325-328 (SEEE), aa 346-349 (TLYE), and at aa 608-611 (SCFE); Tyrosine kinase phosphorylation site at aa 36-43 (RNGIEACY, SEQ ID NO:X), aa 668-675 (RKIQEEYY, SEQ ID NO:X); N-myristoylation sites at aa 38-43 (GIEACY), aa 50-55 (GNGVTI), aa 80-85 (GSYYCM), aa 382-387 (GSWSSE), aa 388-393 (GCELTY), aa 434-439 (GIIISL), aa 480-485 (GINTNT), aa 521-526 (GVIYNK), aa 584-589 (GNLLAF), and at aa 619-624 (GAPRSF); Aspartic acid and asparagine hydroxylation site at 75-86 (CTNTEGSYYCMC, SEQ ID NO:X), EF-hand calcium-binding domain at 153-165 (DLSPTDIITYIEI, SEQ ID NO:X).

Tissue Distribution of SLGP mRNA

This Example describes the tissue distribution of SLGP mRNA, as determined by Northern blot hybridization.

Northern blot hybridizations with the various RNA samples were performed (Clontech Human Multi-tissue Northern I and a human normal and diseased heart tissue northern) under standard conditions and washed under stringent conditions. A 3.2 Kb and a 4.2 Kb mRNA transcript was detected in all tissues tested (heart, brain, placenta, lung, liver, skeletal muscle, kidney, pancreas), with the highest expression in heart. Additionally, these transcripts were found in both normal and diseased hearts.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated nucleic acid molecule selected from the group consisting of:
a) a nucleic acid molecule comprising a nucleotide sequence which is at least 41.8% homologous to the nucleotide sequence of SEQ ID NO:1, SEQ ID NO:3, the DNA insert of the plasmid deposited with ATCC as Accession Number ______, or a complement thereof;
b) a nucleic acid molecule comprising a fragment of at least 488 nucleotides of a nucleic acid comprising the nucleotide sequence of SEQ ID NO:1, SEQ ID NO:3, the DNA insert of the plasmid deposited with ATCC as Accession Number ______, or a complement thereof;
c) a nucleic acid molecule which encodes a polypeptide comprising an amino acid sequence at least about 27.9% homologous to the amino acid sequence of SEQ ID NO:2 or an amino acid sequence encoded by the DNA insert of the plasmid deposited with ATCC as Accession Number ______;
d) a nucleic acid molecule which encodes a fragment of a polypeptide comprising the amino acid sequence of SEQ ID NO:2 or the polypeptide encoded by the DNA insert of the plasmid deposited with ATCC as Accession Number ______, wherein the fragment comprises at least 15 contiguous amino acid residues of the amino acid sequence of SEQ ID NO:2 or the polypeptide encoded by the DNA insert of the plasmid deposited with ATCC as Accession Number ______; and
e) a nucleic acid molecule which encodes a naturally occurring allelic variant of a polypeptide comprising the amino acid sequence of SEQ ID NO:2 or an amino acid sequence encoded by the DNA insert of the plasmid deposited with ATCC as Accession Number ______, wherein the nucleic acid molecule hybridizes to a nucleic acid molecule comprising SEQ ID NO:1 or SEQ ID NO:3, under stringent conditions.

2. The isolated nucleic acid molecule of claim 1 which is selected from the group consisting of:
a) a nucleic acid molecule comprising the nucleotide sequence of SEQ ID NO:1, SEQ ID NO:3, or the DNA insert of the plasmid deposited with ATCC as Accession Number ______, or a complement thereof; and
b) a nucleic acid molecule which encodes a polypeptide comprising the amino acid sequence of SEQ ID NO:2 or an amino acid sequence encoded by the DNA insert of the plasmid deposited with ATCC as Accession Number ______.

3. The nucleic acid molecule of claim 1 further comprising vector nucleic acid sequences.

4. The nucleic acid molecule of claim 1 further comprising nucleic acid sequences encoding a heterologous polypeptide.

5. A host cell which contains the nucleic acid molecule of claim 1.

6. The host cell of claim 5 which is a mammalian host cell.

7. A non-human mammalian host cell containing the nucleic acid molecule of claim 1.

8. An isolated polypeptide selected from the group consisting of:
a) a fragment of a polypeptide comprising the amino acid sequence of SEQ ID NO:2 or the polypeptide encoded by the DNA insert of the plasmid deposited with ATCC as Accession Number ______, wherein the fragment comprises at least 15 contiguous amino acids of SEQ ID NO:2 or the amino acid sequence encoded by the DNA insert of the plasmid deposited with ATCC as Accession Number ______;
b) a naturally occurring allelic variant of a polypeptide comprising the amino acid sequence of SEQ ID NO:2 or an amino acid sequence encoded by the DNA insert of the plasmid deposited with ATCC as Accession Number ______, wherein the polypeptide is encoded by a nucleic acid molecule which hybridizes to a nucleic acid molecule comprising SEQ ID NO:1 or SEQ ID NO:3 under stringent conditions; and
c) a polypeptide which is encoded by a nucleic acid molecule comprising a nucleotide sequence which is at least 41.8% homologous to a nucleic acid comprising the nucleotide sequence of SEQ ID NO:1, SEQ ID NO:3, or the DNA insert of the plasmid deposited with ATCC as Accession Number ______.
d) a polypeptide comprising an amino acid sequence which is at least 27.9% homologous to the amino acid sequence of SEQ ID NO:2, or the polypeptide encoded by the DNA insert of the plasmid deposited with ATCC as Accession Number ______.

9. The isolated polypeptide of claim 8 comprising the amino acid sequence of SEQ ID NO:2 or an amino acid sequence encoded by the DNA insert of the plasmid deposited with ATCC as Accession Number ______.

10. The polypeptide of claim 8, further comprising heterologous amino acid sequences.

11. An antibody which selectively binds to a polypeptide of claim 8.

12. A method for producing a polypeptide selected from the group consisting of:
a) a polypeptide comprising the amino acid sequence of SEQ ID NO:2 or an amino acid sequence encoded by the DNA insert of the plasmid deposited with ATCC as Accession Number ______;
b) a fragment of a polypeptide comprising the amino acid sequence of SEQ ID NO:2 or an amino acid sequence encoded by the DNA insert of the plasmid deposited with ATCC as Accession Number ______ wherein the fragment comprises at least 15 contiguous amino acids of SEQ ID NO:2 or the amino acid sequence encoded by the DNA insert of the plasmid deposited with ATCC as Accession Number ______; and
c) a naturally occurring allelic variant of a polypeptide comprising the amino acid sequence of SEQ ID NO:2 or an amino acid sequence encoded by the DNA insert of the plasmid deposited with ATCC as Accession Number ______, wherein the polypeptide is encoded by a nucleic acid molecule which hybridizes to a nucleic acid molecule comprising SEQ ID NO:1 or SEQ ID NO:3 under stringent conditions;
comprising culturing the host cell of claim 5 under conditions in which the nucleic acid molecule is expressed.

13. A method for detecting the presence of a polypeptide of claim 8 in a sample comprising:
a) contacting the sample with a compound which selectively binds to the polypeptide; and
b) determining whether the compound binds to the polypeptide in the sample to thereby detect the presence of a polypeptide of claim 8 in the sample.

14. The method of claim 13, wherein the compound which binds to the polypeptide is an antibody.

15. A kit comprising a compound which selectively binds to a polypeptide of claim 8 and instructions for use.

16. A method for detecting the presence of a nucleic acid molecule in claim 1 in a sample comprising:
a) contacting the sample with a nucleic acid probe or primer which selectively hybridizes to the nucleic acid molecule; and
b) determining whether the nucleic acid probe or primer binds to a nucleic acid molecule in the sample to thereby detect the presence of a nucleic acid molecule of claim 1 in the sample.

17. The method of claim 16, wherein the sample comprises mRNA molecules and is contacted with a nucleic acid probe.

18. A kit comprising a compound which selectively hybridizes to a nucleic acid molecule of claim 1 and instructions for use.

19. A method for identifying a compound which binds to a polypeptide of claim 8 comprising:
a) contacting the polypeptide, or a cell expressing the polypeptide with a test compound; and
b) determining whether the polypeptide binds to the test compound.

20. The method of claim 19, wherein the binding of the test compound to the polypeptide is detected by a method selected from the group consisting of:
a) detection of binding by direct detection of test compound/polypeptide binding;
b) detection of binding using a competition binding assay; and
c) detection of binding using an assay for SLGP activity.

21. A method for modulating the activity of a polypeptide of claim 8 comprising contacting the polypeptide or a cell expressing the polypeptide with a compound which binds to the polypeptide in a sufficient concentration to modulate the activity of the polypeptide.

22. A method for identifying a compound which modulates the activity of a polypeptide of claim 8 comprising:
a) contacting a polypeptide of claim 8 with a test compound; and
b) determining the effect of the test compound on the activity of the polypeptide to thereby identify a compound which modulates the activity of the polypeptide.
